(19) United States
(12) Patent Application Publication
(10) Pub. No.: US 2018/0371413 A1
(43) Pub. Date: Dec. 27, 2018

(54) PRODUCTS AND METHODS FOR ACTIVATING AND/OR EXPANDING T CELLS

(71) Applicant: CTM@CRC Ltd., Adelaide, South Australia (AU)

(72) Inventors: Simon Barry, Longwood, South Australia (AU); Bahman Delalat, Para Hills, South Australia (AU); Batjargal Gundsambuu, Clovelly Park, South Australia (AU); Jane Frances Harding, Torrensville, South Australia (AU); Dietmar Hutmacher, Belbowrie, Queensland (AU); Maria Elena Juan Pardo, Bowen Hills, Queensland (AU); Nicolas Hans Voelcker, Blackwood, South Australia (AU); Felix Wunner, Paddington, Queensland (AU)

(21) Appl. No.: 15/755,670

(22) PCT Filed: Aug. 26, 2016
(86) PCT No.: PCT/AU2016/050800

§ 371 Date: Feb. 27, 2018

(30) Foreign Application Priority Data
Aug. 28, 2015 (AU) 2015903495

(51) Int. Cl. C12N 5/0783; C07K 16/28; C07K 14/78

(52) U.S. Cl. CPC C12N 5/0637; C07K 16/2809; C07K 16/2818; C07K 14/78; C12N 2501/2302; C12N 2501/505; C12N 2533/52; C12N 2533/50; C12N 2535/00; C12N 2501/51; C12N 2501/515; C12N 2533/30

(57) ABSTRACT

The present disclosure relates to products and methods for activating and/or expanding T cells. Certain embodiments of the present disclosure provide a porous scaffold comprising one or more conjugated T cell stimulatory molecules.

### PRIORITY CLAIM

This application claims priority to Australian provisional patent application number Australian Provisional Patent Application 2015903495 filed on 28 Aug. 2015, the content of which is hereby incorporated by reference.

### FIELD

The present disclosure relates to products and methods for activating and/or expanding T cells.

### BACKGROUND

The ability to activate and/or expand T cells is not only important in understanding T cell biology, but is also important for therapeutic purposes so as to provide increased cell numbers for cell therapies.

In the absence of accessory cells and or exogenous growth factors to activate and expand T cells, it has been recognised that activation and expansion of T cells can be achieved by delivering antigen mimicking signals, by cross linking the T cell receptor and co-stimulatory signals to a T cell population by way of attachment to a solid substrate, such as a bead. In this way, activation and expansion of T cells can be achieved without the need for preparing antigen-presenting cells or antigen.

In many cases, solid substrates utilising stimulatory signals need to be separated from the cells, particularly after expansion of the cells. Ease of separation of cells from a stimulatory substrate is therefore an important consideration in activating and expanding T cells.

For example, beads with stimulatory molecules attached are a frequently used method to activate and expand T cells. However, such technologies provide a challenge to separating the beads from the cells after expansion. One solution to this problem is the use of magnetic beads which can be separated from the cells under the influence of a magnetic field.

A further disadvantage of such bead based technologies is that they typically need to be added as a free component to the cells during the activation and expansion process and as such cannot be incorporated into fixed components used when culturing the cells.

Accordingly, for a variety of reasons there is a need for new reagents and methodologies that can be used to activate and/or expand T cells.

### SUMMARY

Certain embodiments of the present disclosure provide a porous scaffold comprising one or more conjugated T cell stimulatory molecules.

Certain embodiments of the present disclosure provide a method of expanding a T cell, the method comprising exposing a T cell to a porous scaffold comprising one or more conjugated T cell stimulatory molecules and culturing the T cell so as to expand the T cell.

Certain embodiments of the present disclosure provide a composition comprising one or more T cells activated by exposing the one or more T cells to a porous scaffold comprising one or more conjugated T cell stimulatory molecules.

Certain embodiments of the present disclosure provide a composition comprising one or more T cells and a porous scaffold comprising one or more conjugated T cell stimulatory molecules.

Certain embodiments of the present disclosure provide a complex comprising a T cell bound to a porous scaffold comprising one or more conjugated T cell stimulatory molecules.

Certain embodiments of the present disclosure provide a method of producing a porous scaffold for activating a T cell, the method comprising conjugating one or more T cell stimulatory molecules to a porous scaffold.

Other embodiments as described herein.

### BRIEF DESCRIPTION OF THE FIGURES

Exemplary embodiments will be better understood and appreciated in conjunction with the following detailed description of example embodiments taken together with the accompanying figures. It is to be understood that the following description of the figures is for the purpose of describing example embodiments only and is not intended to be limiting with respect to this disclosure.

FIG. 1 shows average fold increase in CD4⁺ lymphocyte number cultured on medical grade poly-ε-caprolactone mPCL electrospun scaffolds, comparing antibody conjugated and bare scaffolds. Scaffolds of fibre spacing 1 mm, 0.5 mm and 0.2 mm were compared. Cell seeding density=1.25×10⁵ per culture. N=3. Error bars show standard error.

FIG. 2 shows viability of CD4⁺ lymphocytes cultured on mPCL electrospun scaffolds, comparing antibody conjugated and bare scaffolds. Scaffolds of fibre spacing 1 mm, 0.5 mm and 0.2 mm were compared. Control cultures without scaffolds were included. Average live and dead cell numbers per culture shown. Cell seeding density=1.25×10⁵ per culture. N=3. Error bars show standard error.

FIG. 3 shows the fold expansion of cells using scaffolds with varying amounts of stimulatory molecules conjugated to the scaffold.

FIG. 4 shows the fold expansion of Treg cells after 7 days using scaffolds with conjugated stimulatory molecules at various cell densities.

FIG. 5 shows fold increase in CD8⁺ cells after 7 days using a scaffold with conjugated stimulatory molecules and CD8⁺ cells seeded at a cell density of 125,000 cells/well as compared to 1:1 beads and CD8⁺ cells at the same density.

FIG. 6 shows that Treg cells respond in vitro to culture on a scaffold and proliferate at a similar rate to Treg cells exposed to CD3/CD28 beads using donor matched input cells. Panel A shows that Treg cell output from the scaffold is equivalent to bead cultures (n=3). Panel B shows analysis of expression levels of FOXP3 and CTLA4 from expanded Treg showing an increase in expression compared with bead expanded donor matched Treg (n=6).

FIG. 7 shows surface phenotype of scaffold and bead expanded Treg cells after 7 days culture and 5 day rest post expansion.

FIG. 8 shows expression of CD25 and FOXP3 protein from scaffold expanded Treg cells is elevated relative to bead expanded donor matched Treg (n=6).

FIG. 9 shows in vitro suppression potency assay using Mixed Lymphocyte Reaction from donor matched Treg pools. Treg expanded using the functionalised scaffolds show increased potency over the limiting dilutions tested compared with bead expanded Tregs (2 unrelated donors).

FIG. 10 shows scaffold activation mediated GFP gene delivery. Photomicrographs show increased green fluorescence in CD4⁺ cell populations transduced on the scaffold, compared with dynal beads. Inset is bright field cell image (2 unrelated donors).

FIG. 11 shows scaffold activation mediated GFP gene delivery: L donor 1, R donor 2. Bead expanded cells shown with dotted line, black line shows scaffold expanded cells. For scaffold expanded cells (% positive) GFP delivery is as efficient as with beads, but the GFP expression levels (MFI) are higher using scaffolds (2 unrelated donors).

FIG. 12 shows functionalised scaffolds expand other T cell populations with equivalent performance. Panel A shows robust CD8⁺ cell expansion on scaffolds compared with Dynal beads n=3. Panel B shows CD8⁺ cell phenotype and purity is retained after expansion with scaffold (2 unrelated donors).

FIG. 13 shows antibody-coated 3D scaffold supports the robust expansion of T lymphocytes. Panel A shows schematic of antibody conjugation to the scaffold. Panel B shows cell microarray platform and that under the conditions tested a desirable CD4⁺ cell expansion is obtained when 40 μg/mL anti-CD3 and anti-CD28 antibody solution is immobilised.

FIG. 14 shows titration of optimal antibody concentration for scaffold coating for CD4⁺ T cell expansion.

FIG. 15 shows CD4⁺ output as a function of scaffold fibre spacing.

FIG. 16 shows CD4⁺ output as a function of number of scaffold layers and scaffold diameter.

FIG. 17 shows mPCL scaffold organisation is critical to expansion. FIG. 14 shows CD4⁺ T cells from six unrelated donors expanded in parallel show reproducibility of output and high viability of expanded T cells.

FIG. 18 shows mean viable cell output from G-Rex 10 cultures comparing dynal bead and scaffold expansion cultures.

FIG. 19 shows maximum viable cell output achieved in G-Rex 10 cultures for dynal bead and scaffold expansion, for two independent donors.

### DETAILED DESCRIPTION

The present disclosure relates to products and methods for activating and/or expanding T cells.

Certain embodiments of the present disclosure are directed to products, methods and kits that have one or more advantages. For example, some advantages of some of the embodiments disclosed herein include one or more of the following: new reagents for activating and/or expanding T cells; a new form of stimulatory substrate for T cells; a new form of substrate for presenting stimulatory molecules to T cells; a methodology for activating and/or expanding T cells that provides ease in separation of cells from the stimulatory substrate; a stimulatory substrate that can be incorporated if desired into cell culture vessels, such as culture flasks and plates; to provide one or more advantages, or to provide a commercial alternative. Other advantages of certain embodiments of the present disclosure are also disclosed herein.

Certain embodiments of the present disclosure provide a porous scaffold comprising one or more conjugated T cell stimulatory molecules.

Certain embodiments of the present disclosure provide a T cell stimulatory substrate comprising a porous scaffold comprising one or more conjugated T cell stimulatory molecules.

The term “porous scaffold” as used herein refers to a solid or a semi-solid substrate having openings or apertures in the substrate to which one or more stimulatory molecules are attached, and includes openings or apertures which allow a cell partially or completely to occupy, and/or openings or apertures through which a cell, or a cluster of cells may pass. Examples of porous scaffolds include a fibrous substrate with openings or apertures between the fibres of the substrate, a mesh with openings or apertures between the network of structural components that make up the mesh, or a solid substrate with holes through the substrate, such as a sponge or foam. Other types of porous scaffold are contemplated.

In certain embodiments, the porous scaffold comprises an ordered scaffold. In certain embodiments, the porous scaffold comprises an organised scaffold. In certain embodiments, the porous scaffold comprises a structured scaffold. In certain embodiments, the scaffold comprises a scaffold with one or more repeating structures.

In certain embodiments, the scaffold comprises a laydown pattern of fibres. In certain embodiments, the laydown pattern comprises a pattern of about 0°/90°. Other suitable laydown patterns comprising variations in the angular arrangement/positioning of fibres are contemplated, and which can be tested for suitability as described herein.

In certain embodiments, the porous scaffold comprises an average or mean pore size of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining pore size are known in the art. In certain embodiments, the pore size comprises the size of the largest cross sectional diameter of a pore.

In certain embodiments, the porous scaffold comprises an average or mean pore size of greater than 100 μm. In certain embodiments, the porous scaffold comprises an average or mean pore size of about 200 μm.

In certain embodiments, the porous scaffold comprises an average or mean pore size in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

In certain embodiments, the porous scaffold comprises a fibrous scaffold.

In certain embodiments, the fibrous scaffold comprises an ordered scaffold. In certain embodiments, the scaffold comprises an organised fibrous scaffold. In certain embodiments, the scaffold comprises a structured fibrous scaffold. In certain embodiments, the scaffold comprises a fibrous scaffold with one or more repeating structures. In certain embodiments, the fibrous scaffold comprises an ordered arrangement of fibres.

In certain embodiments, the pores of a fibrous scaffold comprise the spacing between fibres.

In certain embodiments, the fibrous scaffold comprises an average or mean fibre spacing of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining fibre spacing are known in the art.

In certain embodiments, the fibrous scaffold comprises an average or mean fibre spacing of greater than 100 μm. In certain embodiments, the fibrous scaffold comprises an average or mean fibre spacing of about 200 μm.

In certain embodiments, the fibrous scaffold comprises an average or mean fibre spacing in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

In certain embodiments, the fibrous scaffold comprises fibres with an average or mean diameter of 5 to 20 μm. Methods for determining fibre diameter are known in the art.

In certain embodiments, the fibrous scaffold comprises fibres with an average or mean diameter of 1 μm to 25 μm, 5 μm to 25 μm, 10 μm to 25 μm, 15 μm to 25 μm, 20 to 25 μm, 1 μm to 20 μm, 5 μm to 20 μm, 10 μm to 20 μm, 15 μm to 20 μm, 1 μm to 15 μm, 5 μm to 15 μm, 10 μm to 15 μm, 1 μm to 10 μm, 5 μm to 10 μm, or 1 μm to 5 μm. Other sizes are contemplated.

In certain embodiments, the fibrous scaffold comprises one or more layers of fibrous scaffold. In certain embodiments, the fibrous scaffold comprises a plurality of layers.

In certain embodiments, the fibrous scaffold comprises 5 to 25 layers, 10 to 25 layers, 15 to 25 layers, 20 to 25 layers, 5 to 20 layers, 10 to 20 layers, 15 to 20 layers, 5 to 15 layers, 10 to 15 layers, or 5 to 10 layers. Other numbers of layers are contemplated.

In certain embodiments, the fibrous scaffold comprises 5 to 20 layers.

In certain embodiments, the fibrous scaffold comprises a substantial alignment of pores between layers.

In certain embodiments, at least 90%, at least 80%, at least 70%, at least 60%, at least 50%, at least 40%, at least 30%, at least 20% of the pore area is aligned through the scaffold. In certain embodiments, at least 90%, at least 80%, at least 70%, at least 60%, at least 50%, at least 40%, at least 30%, at least 20% of the pore area is overlapping through the scaffold.

In certain embodiments, at least 50% of the pore area is aligned through the scaffold. In certain embodiments, at least 50% of the pore area is overlapping through the scaffold.

In certain embodiments, the porous scaffold comprises a mesh, a mat, a woven matrix, a cloth and/or a sponge. Other types of porous scaffold are contemplated.

In certain embodiments, the porous scaffold is produced by electrospinning. In certain embodiments, the porous scaffold is produced by a method using electrospinning. Examples of electrospinning include coaxial electrospinning, emulsion electrospinning, and melt electrospinning. Methods for performing electrospinning are known in the art.

In certain embodiments, the porous scaffold is produced by melt electrospinning. In certain embodiments, the porous scaffold is produced by a method using melt electro spinning.

In certain embodiment, the porous scaffold comprises a synthetic scaffold. In certain embodiments, the porous scaffold comprises a natural scaffold or a scaffold derived from a natural product.

In certain embodiments, the porous scaffold comprises electrospun fibres. Methods for electrospinning are known in the art.

In certain embodiments, the porous scaffold comprises one or more of a polylactide polymer, a polyglycolic acid polymer, a polycaprolactone polymer, a poly (amino acid alkyl ester) phosphazene polymer, a poly(caprolactone co-ethyl ethylene phosphate) polymer, a polycarbonate polymer, a polyethyleneimine polymer, a polyethyleneglycol polymer, a polyurethane polymer, and a poly vinyl alcohol polymer. Other types of polymers are contemplated.

In certain embodiments, the porous scaffold comprises an electrospun polycaprolactone scaffold.

In certain embodiments, the porous scaffold is sterilisable. In certain embodiments, the porous scaffold is shelf stable.

The term “stimulatory molecule” as used herein refers to a molecule that alone, or in combination with one or more other molecules, activates a T cell.

In certain embodiments, the one or more T cell stimulatory molecules comprise a binding molecule for one or more of CD3, CD28, CD5, CD2, CD44, CD137, CD9, CD278, alpha integrin and a beta integrin and isoforms thereof, and any combination thereof. Binding molecules for the aforementioned molecules are available or may be produced by a known method, and includes parts, fragments or regions of binding molecules that have the capacity to bind the target

In certain embodiments, the one or more T cell stimulatory molecules comprise a binding molecule for CD3. The CD3 T-cell co-receptor is a protein complex and is composed of four distinct chains. In mammals, the complex contains a CD3γ chain, a CD36 chain, and two CD3c chains. The chains associate with the T-cell receptor (TCR) and the CD3 ξ-chain to form a TCR complex. CD3 in humans and other species may be readily identified by a person skilled in the art.

In certain embodiments, the one or more T cell stimulatory molecules comprise a binding molecule for CD28. CD28 is a protein expressed on T cells and the UniProtKB/Swiss-Prot accession number for the human protein is P10747. CD28 in other species, and homologues, paralogues, orthologues and/or variants thereof, may all be readily identified (for example by using the BLAST suite of algorithms) and are included within the scope of the present disclosure.

In certain embodiments, the one or more T cell stimulatory molecules comprise a binding molecule for CD5. CD5 is a protein expressed on a subset of IgM-secreting B cells (B-1 cells). The UniProtKB/Swiss-Prot accession number for the human protein is P06127. CD5 in other species, and homologues, paralogues, orthologues and/or variants thereof, may all be readily identified and are included within the scope of the present disclosure.

In certain embodiments, the one or more T cell stimulatory molecules comprise a binding molecule for CD2. CD2 is a cell adhesion molecule found on the surface of T cells and natural killer (NK) cells. The UniProtKB/Swiss-Prot accession number for the human protein is P06729. CD2 in other species, and homologues, paralogues, orthologues and/or variants thereof, may all be readily identified and are included within the scope of the present disclosure.

In certain embodiments, the one or more T cell stimulatory molecules comprise a binding molecule for CD44. CD44 is a cell-surface glycoprotein. The UniProtKB/Swiss-Prot accession number for the human protein is P16070. CD44 in other species, and homologues, paralogues, orthologues and/or variants thereof, may all be readily identified and are included within the scope of the present disclosure.

In certain embodiments, the one or more T cell stimulatory molecules comprise a binding molecule for CD137. CD137 is a member of the tumour necrosis factor (TNF) receptor family. The UniProtKB/Swiss-Prot accession number for the human protein is Q07011. CD137 in other species, and homologues, paralogues, orthologues and/or variants thereof, may all be readily identified and are included within the scope of the present disclosure.

In certain embodiments, the one or more T cell stimulatory molecules comprise a binding molecule for CD9. CD9 is a cell surface glycoprotein. The UniProtKB/Swiss-Prot accession number for the human protein is P21926. CD9 in other species, and homologues, paralogues, orthologues and/or variants thereof, may all be readily identified and are included within the scope of the present disclosure.

In certain embodiments, the one or more T cell stimulatory molecules comprise a binding molecule for CD278. CD278 is a CD28-superfamily costimulatory molecule that is expressed on activated T cells. The UniProtKB/Swiss-Prot accession number for the human protein is Q9Y6W8. CD278 in other species, and homologues, paralogues, orthologues and/or variants thereof, may all be readily identified and are included within the scope of the present disclosure.

In certain embodiments, the one or more T cell stimulatory molecules comprise a binding molecule for CD49. CD49a, b, c, d, e and f are examples of members of the integrin alpha family (see for example Genecards ID GC17P050055, GC12M054396, GC02P181456, GC05P052989, GC05P052788, and GC02P172427). These molecules facilitate cell attachment and co-stimulation, either alone, or in association with members of the beta integrin family including CD29 (see UniProtKB—P05556 ITB1 HUMAN), CD18 (see UniProtKB—P05107 ITB2 HUMAN), CD 61 (see UniProtKB—P05106 ITB3 HUMAN) and CD104 (see UniProtKB—P16144 ITB4 HUMAN).

In certain embodiments, the one or more T cell stimulatory molecules comprise a binding molecule for CD3.

In certain embodiments, the one or more T cell stimulatory molecules comprise a binding molecule for CD3 and a binding molecule for one or more of CD28, CD5, CD2, CD44, CD137, CD9, CD278, alpha integrin and a beta integrin.

In certain embodiments, the one or more T cell stimulatory molecules comprise a binding molecule for CD3 and/or a binding molecule for CD28.

In certain embodiments, the porous scaffold comprises one T cell stimulatory molecule. In certain embodiments, the porous scaffold comprises two T cell stimulatory molecules. In certain embodiments, the porous scaffold comprises two or more T cell stimulatory molecules.

In certain embodiments, the porous scaffold comprises two or more binding molecules for CD3, CD28, CD5, CD2, CD44, CD137, CD9, CD278, an integrin alpha and an integrin beta.

In certain embodiments, the one or more T cell stimulatory molecules comprise a binding molecule for CD3 and/or a binding molecule for CD28.

In certain embodiments, the one or more stimulatory molecules comprises an antibody, a receptor, a small molecule, a nucleic acid, an aptamer, a polypeptide, a protein, a glycoprotein, a ligand or a ligand mimetic. Other types of agents are contemplated.

In certain embodiments, the one or more stimulatory molecules comprise an antibody.

The term “antibody” as used herein refers to an immunoglobulin molecule, or a part thereof, with the ability to bind an antigenic region of another molecule, and includes monoclonal antibodies, polyclonal antibodies, multivalent antibodies, chimeric antibodies, multispecific antibodies, diabodies and fragments or parts of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more Complementarity Determining Regions (CDRs).

In this regard, an immunoglobulin is a tetrameric molecule, each tetramer being composed of two identical pairs of polypeptide chains, each pair having one light chain and one heavy chain. The amino-terminal portion of each chain includes a variable region of about 100 to 110 or more amino acids that is primarily responsible for antigen recognition. The carboxy-terminal portion of each chain defines a constant region primarily responsible for effector function. Human light chains are classified as K and λ light chains. Heavy chains are classified as μ, Δ, γ, α, or ε and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. The variable regions of each light/heavy chain pair form the antibody binding site, with the result that an intact immunoglobulin has two binding sites. The variable regions further include hypervariable regions that are directly involved in formation of the antigen binding site. These hypervariable regions are usually referred to as Complementarity Determining Regions (CDR). The intervening segments are referred to as Framework Regions (FR). In both light and heavy chains there are three CDRs (CDR-I to CDR-3) and four FRs (FR-I to FR-4).

In certain embodiments, the antigen-binding part or fragment comprises a Fab, Fab′, F(ab′)₂, Fd, Fv, a single-chain antibody (scFv), a chimeric antibody, a diabody or a polypeptide that contains at least a portion of an immunoglobulin that is sufficient to confer specific antigen binding.

A Fab fragment is a monovalent fragment consisting of the VL, VH, CL and CH I domains. A F(ab′)₂ fragment is a bivalent fragment including two Fab fragments linked by a disulphide bridge at the hinge region. A Fd fragment consists of the VH and CH I domains. A Fv fragment consists of the VL and VH domains of a single arm of an antibody. A dAb consists of a VH domain. A single chain antibody (scFv) is an antibody in which VL and VH regions are paired to form a monovalent molecule via a synthetic linker that enable them to be made as a single protein chain. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

Antibody fragments or parts that contain specific binding sites may be generated by a known method. Methods for producing antigen-binding fragments or portions of antibodies are known in the art, for example as described in “Antibody Engineering: Methods and Protocols” (2004) ed. by B.K.C. Lo Humana Press, herein incorporated by reference; and “Antibody Engineering: A Practical Approach” (1996) ed. by J. McCafferty, H. R. Hoogenboom and DJ. Chriswell Oxford University Press, herein incorporated by reference. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity, as described for example in Huse, W. D. et al. (1989) Science 254: 1275-1281, herein incorporated by reference.

Antibodies may be commercially available or may be generated using known methods. For the production of antibodies, various hosts including goats, rabbits, rats, mice, humans, and others, may be immunised by injection with an appropriate antigen. Depending on the host species, various adjuvants may be used to increase an immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol. Adjuvants are commercially available.

In certain embodiments, the antibody is a polyclonal antibody. A polyclonal antibody is a mixture of antibodies having different antigen specificities. Methods for producing and isolating polyclonal antibodies are known. In general, polyclonal antibodies are produced from B-lymphocytes. Typically polyclonal antibodies are obtained directly from an immunised subject, such as an immunised animal.

In certain embodiments, the antibody is a monoclonal antibody. Monoclonal antibodies may be prepared using a technique that provides for the production of antibody molecules by continuous isolated cells in culture. These include, but are not limited to, the hybridoma technique, the human B-cell hybridoma technique, and the EBV-hybridoma technique. Methods for the preparation of monoclonal antibodies include for example Kohler et al. (1975) Nature 256:495-497, herein incorporated by reference; Kozbor et al. (1985) J. Immunol. Methods 81:31-42, herein incorporated by reference; Cote et al. (1983) Proc. Natl. Acad. Sci. 80:2026-2030, herein incorporated by reference; and Cole et al. (1984) Mol. Cell Biol. 62: 109-120, herein incorporated by reference.

In certain embodiments, the antibody and/or an antigen binding fragment or part thereof comprises an isolated antibody. Methods for producing and isolating polyclonal and monoclonal antibodies are known.

In certain embodiments, the antibody as described herein has an isotype selected from the group consisting of IgG1, IgG2a, IgG2b, IgG3, IgM and IgA. Determination of the isotype of an antibody may be by a known method.

In certain embodiments, the antibody and/or an antigen binding fragment or part thereof is a mouse antibody and/or an antigen binding fragment or part thereof, a human antibody and/or an antigen binding fragment or part thereof, or a humanised antibody and/or an antigen binding fragment or part thereof.

Humanised antibodies, or antibodies adapted for non-rejection by other mammals, may be produced by a suitable method known in the art, including for example resurfacing or CDR grafting. In resurfacing technology, molecular modelling, statistical analysis and mutagenesis are combined to adjust the non-CDR surfaces of variable regions to resemble the surfaces of known antibodies of the target host. Strategies and methods for the resurfacing of antibodies, and other methods for reducing immunogenicity of antibodies within a different host are known, for example as described in U.S. Pat. No. 5,639,641. Humanised forms of the antibodies may also be made by CDR grafting, by substituting the complementarity determining regions of, for example, a mouse antibody, into a human framework domain.

Methods for humanising antibodies are known. For example, the antibody may be generated as described in U.S. Pat. No. 6,180,370, herein incorporated by reference; WO 92/22653, herein incorporated by reference; Wright et al. (1992) Critical Rev. in Immunol. 12(3,4): 125-168, herein incorporated by reference; and Gu et al. (1997) Thrombosis and Hematocyst 77(4):755-759), herein incorporated by reference.

Humanised antibodies typically have constant regions and variable regions other than the complementarity determining regions (CDRs) derived substantially or exclusively from a human antibody and CDRs derived substantially or exclusively from the non-human antibody of interest.

Techniques developed for the production of “chimeric antibodies”, for example the splicing of mouse antibody genes to human antibody genes to obtain a molecule with appropriate antigen specificity and biological activity, may be performed by a suitable method. For example, chimeric antibodies may be produced as described in Morrison, S. L. et al. (1984) Proc. Natl. Acad. Sci. 81:6851-6855, herein incorporated by reference; Neuberger, M. S. et al. (1984) Nature 312:604-608, herein incorporated by reference; and Takeda, S. et al. (1985) Nature 314:452-454, herein incorporated by reference.

Immunoassays may be used for screening to identify antibodies and/or antigen binding fragments thereof having the desired specificity. Protocols for competitive binding or immunoradiometric assays using either polyclonal or monoclonal antibodies are known.

Antibody molecules and antigen binding fragments or parts thereof may also be produced recombinantly by methods known in the art, for example by expression in E. coli expression systems. For example, a method for the production of recombinant antibodies is as described in U.S. Pat. No. 4,816,567, herein incorporated by reference. Antigen binding fragments or parts may also be produced by phage display technologies, which are known.

In certain embodiments, the antibody has an isotype selected from the group consisting of IgG1, IgG2a, IgG2b, IgG3, IgM and IgA.

In certain embodiments, the antibody is a monoclonal antibody and/or an antigen binding fragment or part thereof.

In certain embodiments, the antibody is a human antibody or a humanised antibody.

In certain embodiments, the one or more T cell stimulatory molecules comprise an antibody (or an antigen binding fragment or part thereof) to one or more of CD3, CD28, CD5, CD2, CD44, CD137, CD9, CD278, an alpha integrin or a beta integrin and isoforms thereof. In certain embodiments, the one or more T cell stimulatory molecules comprise one or more of an anti-CD3 antibody, an anti-CD28 antibody, an anti-CD5 antibody, an anti-CD2 antibody, an anti-CD44 antibody, an anti-CD137 antibody, an anti-CD9 antibody, an anti-CD278 antibody, an anti-integrin alpha antibody and an anti-integrin beta antibody, and/or an antigen binding fragment or part thereof. Antibodies to the aforementioned molecules are either commercially available or may be produced by a method known in the art.

In certain embodiments, the one or more T cell stimulatory molecules comprise an anti-CD3 antibody and/or an anti-CD28 antibody, and/or an antigen binding fragment or part thereof. Anti-CD3 antibodies and anti-CD28 antibodies may be produced by a method known in the art or are commercially available, such as anti-human CD3 (Affymetrix eBioscence; Cat. No: 16-0039-81) and anti-human CD28 (BD Pharmingen Cat. No: 555725).

In certain embodiments, the one or more T cell stimulatory molecules comprise one or more of an anti-human CD3 antibody, an anti-human CD28 antibody, an anti-human CD5 antibody, an anti-human CD2 antibody, an anti-human CD44 antibody, an anti-human CD137 antibody, an anti-human CD9 antibody, an anti-human CD278 antibody, an anti-human integrin alpha antibody and an anti-human integrin beta antibody, and/or an antigen binding fragment or part thereof.

In certain embodiments, the one or more T cell stimulatory molecules comprise an anti-human CD3 antibody and/or an anti-human CD28 antibody, and/or an antigen binding fragment or part of either or both of the aforementioned antibodies.

In certain embodiments, the one or more T cell stimulatory molecules comprise an antibody raised to antigen from the same species as that for which the T cells are to be activated and/or expanded. In certain embodiments, the one or more T cell stimulatory molecules comprise an antibody raised to antigen from a different species as that for which the T cells are to be activated and/or expanded.

In certain embodiments, a binding molecule for an integrin comprises fibronectin and/or a fragment, part or a derivative thereof. For example, a fragment of fibronectin is retronectin, and derivatives of fibronectin include fibronectin RGD and fibronectin EILDV (a splice variant). In certain embodiments, the one or more T cell stimulatory molecules comprise fibronectin and/or a fragment, part or a derivative thereof.

In certain embodiments, the porous scaffold comprises a porous electrospun polycaprolactone scaffold presenting anti CD3 and anti CD28 monoclonal antibodies, and/or an antigen binding fragment or part thereof.

In certain embodiments, the conjugation of the one or more T cell co-stimulatory molecules comprises direct or indirect attachment to the porous scaffold.

In certain embodiments, the one or more T cell stimulatory molecules are directly or indirectly attached to the porous scaffold.

In certain embodiments, the one or more T cell stimulatory molecules are non-covalently attached to the porous scaffold. In certain embodiments, the porous scaffold comprises one or more non-covalently attached T cell stimulatory molecules. Methods for non-covalent attachment are known in the art. For example, an immobilisation agent and a capture group may be used for attachment to the porous scaffold. In certain embodiments, the immobilisation agent and the capture group comprise a binding pair, such as biotin and streptavidin, or a ligand and its receptor. Immobilisation agents and capture agents are known in the art.

In certain embodiments, the one or more T cell stimulatory molecules are directly or indirectly covalently linked to the porous scaffold. Methods for covalent attachment/linking are known in the art. For example, linker groups may be used for covalently attachment to the porous scaffold. Linkers are known in the art. Covalent attachment provides advantages with respect to non-covalent attachment, such as stability.

In certain embodiments, the porous scaffold is functionalised for conjugation of the one or more one or more T cell stimulatory molecules. Methods for functionalisation are known in the art.

For example, the porous scaffold may be an epoxy functionalised substrate or surface.

In certain embodiments, the porous scaffold is functionalised for direct or indirect attachment of the one or more one or more T cell stimulatory molecules.

In certain embodiments, the porous scaffold is functionalised by a method comprising plasma polymerisation. In certain embodiments, the porous scaffold is treated by plasma polymerisation in the presence of a monomer to functionalise the scaffold. Methods for performing plasma polymerisation are known in the art. Examples of monomers include allyl glycidyl ether, allylamine, heptylamine, acrylic acid, 1,7 octadiene and ethanol. Other monomers are contemplated.

For example, the porous scaffold may be functionalised using plasma polymerisation of allyl glycidyl ether to produce a reactive epoxy-functionalised surface. Coating using such plasma polymerisation is broadly applicable to most surfaces.

In certain embodiments, the one or more T cell stimulatory molecules are directly or indirectly covalently linked to the porous scaffold via a plasma polymerised functional group, such as a plasma polymerised epoxy group.

In certain embodiments, the one or more T cell stimulatory molecules are attached to the porous scaffold via a linker. In certain embodiments, the porous scaffold comprises one or more T cell stimulatory molecules attached via a linker. Examples of linkers include carboxyl-to-amine linkers, such as carbodiimides, amine-reactive linkers such as NHS esters and imidoesters, sulfhydryl-reactive linkers such as maleimides, haloacetyls and pyridyldisulfides, carbonyl-reactive linkers such as hydrazides and alkoxyamines, photoreactive linkers such as aryl azides and diazirines, chemoselective ligation, such as Staudinger reagent pairs. In certain embodiments, the linker comprises a semicarbazide linker.

In certain embodiments, the stimulatory molecule comprises an antibody (and/or a fragment part thereof) and the antibody is attached to the porous scaffold. In certain embodiments, the stimulatory molecule comprises an antibody and the antibody is attached to the porous scaffold via the Fc chain of the antibody. In certain embodiments, the antibody is attached to the porous scaffold via a carbohydrate group on the antibody. In certain embodiments, the antibody comprises an oxidised carbohydrate group. In certain embodiments, the antibody is attached via an oxidised carbohydrate group to the porous scaffold. Methods for attaching antibodies to a scaffold/substrate are known in the art, for example as described in Secret E. et al (2013) Advanced Healthcare Materials Volume 2, Issue 5, pages 718-727.

In certain embodiments, the porous scaffold is treated with, or exposed to, the stimulatory molecule so as to attach the stimulatory molecule to the scaffold.

In certain embodiments, the functionalised porous scaffold is treated with a concentration of a stimulatory molecule at a concentration of 1 μg/ml or greater, 5 μg/ml or greater, 10 μg/ml or greater, 20 μg/ml or greater, 40 μg/ml or greater, 80 μg/ml or greater, 100 μg/ml or greater, 200 μg/ml or greater, or 500 μg/ml or greater, Other concentrations are contemplated.

In certain embodiments, the functionalised porous scaffold is treated with a concentration of a stimulatory molecule at a concentration of 1 to 200 μg/ml, 10 to 200 μg/ml, 20 to 200 μg/ml, 40 to 200 μg/ml, 80 to 200 μg/ml, 100 to 200 μg/ml, 1 to 100 μg/ml, 10 to 100 μg/ml, 20 to 100 μg/ml, 40 to 100 μg/ml, 80 to 100 μg/ml, 1 to 80 μg/ml, 10 to 80 μg/ml, 20 to 80 μg/ml, 40 to 80 μg/ml, 1 to 40 μg/ml, 10 to 40 μg/ml, 20 to 40 μg/ml, and 1 to 20 μg/ml. Other concentrations are contemplated.

Certain embodiments of the present disclosure provide a method of activating a T cell, using a porous scaffold with one or more conjugated T cell stimulatory molecules as described herein. Methods of determining the activation of T cells are known in the art.

In certain embodiments, the porous scaffold as described herein is used to activate T cells, to expand T cells, to present one or more stimulatory molecules, to stimulate proliferation of T cells, to stimulate proliferation of one or more T cell sub-populations, and/or for the activation and expansion of T cells to provide cell numbers required for adoptive cell transfer therapies to be achieved.

Certain embodiments of the present disclosure provide a method of activating a T cell, the method comprising exposing a T cell to a porous scaffold comprising one or more conjugated T cell stimulatory molecules, and thereby activating the T cell.

The term “exposing”, and related terms such as “expose” and “exposure”, as used herein refers to directly and/or indirectly contacting and/or treating a cell with a scaffold or agent as described herein.

T cells are as described herein. In certain embodiments, the T cell comprises a CD4⁺ T cell, a CD8⁺ T cell (a killer T cell), a CD3⁺ T cell, a CD4⁺CD25⁺ T cell (a regulatory T cell), a chimeric antigen receptor expressing T cell, a natural killer cell or a tumour infiltrating lymphocyte. In certain embodiments, the T cells comprise a T cell sub-population. Other types of T cells are contemplated.

Methods for obtaining T cells of specific types are known in the art. Isolation of human CD3⁺ T cells may be accomplished, for example, from human peripheral blood mononuclear cells, for example using Dynabeads® FlowComp™ Human CD3 Kit (Cat #11365D). Isolation of human regulatory T cells may be accomplished, for example, by firstly indirectly magnetically labelling cells with a cocktail of biotin-conjugated antibodies against one or more of CD8, CD14, CD15, CD16, CD19, CD36, CD56, CD123, TCRγ/δ and CD235a (glycophorin A) and anti-biotin beads. The labelled cells may then subsequently be depleted over a column. In the second step, the flow-through fraction of pre-enriched CD4⁺ T cells may be labelled with CD25 beads for subsequent positive selection of CD4⁺CD25⁺ regulatory T cells, for example using a CD4⁺CD25⁺ Regulatory T Cell Isolation Kit (Miltenyl Biotec; Cat #130-091-301). Natural killer cells may be isolated, for example, using a Miltenyl Biotec NK Cell Isolation Kit (Cat #130-092-657). Chimeric antigen receptor expressing T cells and tumour infiltrating lymphocytes are described, for example, in Wang X. and Rivière I. (2015) Cancer Gene Therapy 22(2).

Methods for assessing the activation of a T cell are known in the art, for example by using markers such as CD44 and CD137.

In certain embodiments, the porous scaffold comprises an average or mean pore size of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining pore size are known in the art. In certain embodiments, the pore size comprises the size of the largest cross sectional diameter of a pore.

In certain embodiments, the porous scaffold comprises an average or mean pore size of greater than 100 μm. In certain embodiments, the porous scaffold comprises an average or mean pore size of about 200 μm.

In certain embodiments, the porous scaffold comprises an average or mean pore size in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

Examples of porous scaffolds are as described herein.

In certain embodiments, the porous scaffold comprises a fibrous scaffold.

In certain embodiments, the pores of a fibrous scaffold comprise the spacing between fibres.

In certain embodiments, the fibrous scaffold comprises an average or mean fibre spacing of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining fibre spacing are known in the art.

In certain embodiments, the fibrous scaffold comprises an average or mean fibre spacing of greater than 100 μm. In certain embodiments, the fibrous scaffold comprises an average or mean fibre spacing of about 200 μm.

In certain embodiments, the fibrous scaffold comprises an average or mean fibre spacing in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

In certain embodiments, the fibrous scaffold comprises fibres with an average or mean diameter of 5 to 20 μm. Methods for determining fibre diameter are known in the art.

In certain embodiments, the fibrous scaffold comprises fibres with an average or mean diameter of 1 μm to 25 μm, 5 μm to 25 μm, 10 μm to 25 μm, 15 μm to 25 μm, 20 to 25 μm, 1 μm to 20 μm, 5 μm to 20 μm, 10 μm to 20 μm, 15 μm to 20 μm, 1 μm to 15 μm, 5 μm to 15 μm, 10 μm to 15 μm, 1 μm to 10 μm, 5 μm to 10 μm, or 1 μm to 5 μm. Other sizes are contemplated.

In certain embodiments, the fibrous scaffold comprises one or more layers of fibrous scaffold. In certain embodiments, the fibrous scaffold comprises a plurality of layers.

In certain embodiments, the fibrous scaffold comprises 5 to 25 layers, 10 to 25 layers, 15 to 25 layers, 20 to 25 layers, 5 to 20 layers, 10 to 20 layers, 15 to 20 layers, 5 to 15 layers, 10 to 15 layers, or 5 to 10 layers. Other numbers of layers are contemplated.

In certain embodiments, the fibrous scaffold comprises 5 to 20 layers.

In certain embodiments, the method comprises activating a T cell sub-population.

Certain embodiments of the present disclosure provide a T cell activated by a method as described herein.

In certain embodiments, the activated T cell comprises a plurality of cells. In certain embodiments, the activated T cells form part of a population of T cells.

Certain embodiments of the present disclosure provide a population of cells comprising T cells activated by a method as described herein.

Certain embodiments of the present disclosure provide a population of T cells activated by a method as described herein.

In certain embodiments, the activated T cells comprise at least 1%, 2%, 5%, 10%, 20%, 30%, 40%, 50%, 60%, 70%, 80%, 90%, 95% or 99% of the total cells in the population. Other levels of activation are contemplated.

In certain embodiments, the population comprises a T cell sub-population.

Certain embodiments of the present disclosure provide a composition comprising one or more T cells activated by a method as described herein.

Certain embodiments of the present disclosure provide a composition comprising one or more T cells activated by exposing the one or more T cells to a porous scaffold comprising one or more conjugated T cell stimulatory molecules.

T cells, porous scaffolds and T cell stimulatory molecules are as described herein.

In certain embodiments, the composition is a therapeutic composition.

Methods for producing compositions of T cells for therapeutic delivery are known in the art. For example, the T cells may be formulated in a suitable liquid carrier for administration.

Certain embodiments of the present disclosure provide a method of expanding a T cell(s) using a porous scaffold comprising one or more conjugated T cell stimulatory molecules as described herein.

Certain embodiments of the present disclosure provide a method of expanding a T cell, the method comprising exposing the T cell to a porous scaffold comprising one or more conjugated T cell stimulatory molecules and culturing the T cell so as to expand the T cell.

Examples of T cells are as described herein. In certain embodiments, the T cell comprises a CD4⁺ T cell (including helper T cells), a CD8+ T cell (a killer T cell), a CD3+ T cell, a CD4⁺CD25⁺ T cell (a regulatory T cell), a chimeric antigen receptor T expressing cell, a natural killer cell or a tumour infiltrating lymphocyte. In certain embodiments, the T cells comprise a T cell sub-population. Other types of T cells are contemplated.

Methods for obtaining T cells are as described herein.

Stimulatory molecules are as described herein. In certain embodiments, the one or more T cell stimulatory molecules comprises a binding molecule for one or more of CD3, CD28, CD5, CD2, CD44, CD137, CD9, CD278, an alpha integrin and a beta integrin and isoforms thereof. In certain embodiments, the one or more T cell stimulatory molecules comprise a binding molecule for CD3 and/or a binding molecule for CD28.

In certain embodiments, the one or more T cell stimulatory molecules comprise an antibody and/or antigen binding fragment or part thereof.

In certain embodiments, the one or more T cell stimulatory molecules comprise an anti-CD3 antibody and/or an anti-CD28 antibody.

In certain embodiments, the one or more T cell stimulatory molecules comprise fibronectin and/or a fragment or derivative thereof.

Methods of conjugation of a stimulatory molecule(s) to a porous scaffold are as described herein. In certain embodiments, the one or more T cell stimulatory molecules are directly or indirectly covalently linked to the porous scaffold.

Examples of porous scaffolds are as described herein.

In certain embodiments, the porous scaffold comprises an average or mean pore size of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining pore size are known in the art. In certain embodiments, the pore size comprises the size of the largest cross sectional diameter of a pore.

In certain embodiments, the porous scaffold comprises an average or mean pore size of greater than 100 μm. In certain embodiments, the porous scaffold comprises an average or mean pore size of about 200 μm.

In certain embodiments, the porous scaffold comprises an average or mean pore size in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

In certain embodiments, the porous scaffold comprises a mesh, a mat, a woven matrix and/or a sponge.

In certain embodiments, the porous scaffold comprises a fibrous scaffold.

In certain embodiments, the fibrous scaffold comprises an ordered scaffold. In certain embodiments, the scaffold comprises an organised fibrous scaffold. In certain embodiments, the scaffold comprises a structured fibrous scaffold. In certain embodiments, the scaffold comprises a fibrous scaffold with one or more repeating structures. In certain embodiments, the fibrous scaffold comprises an ordered arrangement of fibres.

In certain embodiments, the scaffold comprises a laydown pattern of fibres. In certain embodiments, the laydown pattern comprises a pattern of about 0°/90°. Other suitable laydown patterns comprising variations in the angular arrangement/positioning of fibres are contemplated.

In certain embodiments, the fibrous scaffold comprises an average or mean fibre spacing of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Other sizes are contemplated. Methods for determining fibre spacing are known in the art.

In certain embodiments, the fibrous scaffold comprises an average or mean fibre spacing of greater than 100 μm. In certain embodiments, the fibrous scaffold comprises an average or mean fibre spacing of about 200 μm.

In certain embodiments, the fibrous scaffold comprises an average or mean fibre spacing in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm. Other ranges are contemplated.

In certain embodiments, the fibrous scaffold comprises fibres with an average or mean diameter of 5 to 20 μm. Methods for determining fibre diameter are known in the art.

In certain embodiments, the fibrous scaffold comprises fibres with an average or mean diameter of 1 μm to 25 μm, 5 μm to 25 μm, 10 μm to 25 μm, 15 μm to 25 μm, 20 to 25 μm, 1 μm to 20 μm, 5 μm to 20 μm, 10 μm to 20 μm, 15 μm to 20 μm, 1 μm to 15 μm, 5 μm to 15 μm, 10 μm to 15 μm, 1 μm to 10 μm, 5 μm to 10 μm, or 1 μm to 5 μm. Other sizes are contemplated.

In certain embodiments, the fibrous scaffold comprises one or more layers of fibrous scaffold. In certain embodiments, the fibrous scaffold comprises a plurality of layers.

In certain embodiments, the fibrous scaffold comprises 5 to 25 layers, 10 to 25 layers, 15 to 25 layers, 20 to 25 layers, 5 to 20 layers, 10 to 20 layers, 15 to 20 layers, 5 to 15 layers, 10 to 15 layers, or 5 to 10 layers. Other numbers of layers are contemplated.

In certain embodiments, the fibrous scaffold comprises 5 to 20 layers.

In certain embodiments, the porous scaffold comprises electrospun fibres. In certain embodiments, the porous scaffold comprises melt electrospun fibres.

In certain embodiments, the porous scaffold comprises one or more of a polylactide polymer, a polyglycolic acid polymer, a polycaprolactone polymer, a poly (amino acid alkyl ester) phosphazene polymer, a poly(caprolactone co-ethyl ethylene phosphate) polymer, a polycarbonate polymer, a polyethyleneimine polymer, a polyethyleneglycol polymer, a polyurethane polymer, and a poly vinyl alcohol polymer. Other polymers are contemplated.

In certain embodiments, the culturing of the T cell(s) comprises culturing with one or more growth factors. In certain embodiments, the culturing of the T cell(s) comprises culturing without growth factors.

In certain embodiments, the culturing of the T cell(s) comprises culturing in the presence of the porous scaffold. In certain embodiments, the culturing of the T cells in the presence of the porous scaffold comprises culturing the T cells in the presence of the porous scaffold during substantially the entire process of expansion of the cells. In certain embodiments, the culturing of the T cells in the presence of the porous scaffold comprises culturing the T cells in the presence of the porous scaffold during part of the process of expansion of the cells.

In certain embodiments, the method comprises expanding the cells by 5 fold or more, 10 fold or more, 15 fold or more, 20 fold or more, 30 fold or more, 40 fold or more, 50 fold or more, 60 fold or more, 70 fold or more, 80 fold or more, 90 fold or more, or 100 fold or more. Methods for assessing the expansion of a T cell are known in the art. Other levels of expansion are contemplated.

In certain embodiments, the method comprises expanding 1×10⁴ cells or more, 5×10⁴ cells or more, 1×10⁵ cells or more, 5×10⁵ cells or more, or 1×10⁶ cells or more.

In certain embodiments, the expansion of the T cells enables sufficient cell numbers required for adoptive cell transfer therapies to be achieved. In certain embodiments, the expansion provides at least 10⁸ cells, at least 10⁹ cells, or at least 10¹⁰ cells, for cell transfer therapy

Certain embodiments of the present disclosure provide T cells expanded by a method as described herein.

In certain embodiments, the expanded T cells comprise a plurality of cells. In certain embodiments, the expanded T cells form part of a population of T cells.

Certain embodiments of the present disclosure provide a population of T cells expanded by a method as described herein.

In certain embodiments, the expanded T cells comprises at least 1%, 2%, 5%, 10%, 20%, 30%, 40%, 50%, 60%, 70%, 80%, 90%, 95% or 99% of the total cells in the population.

In certain embodiments, the method comprises culturing the T cell, and/or expanded cells therefrom, in the presence of accessory cells and/or an endogenous and/or exogenous growth factor(s). Methods for culturing cells, including culturing with accessory cells and/or endogenous and/or exogenous agents, are known in the art. In certain embodiments, the method comprises culturing the T cell, and/or expanded cells therefrom, in the absence of accessory cells and/or an exogenous growth factor(s).

Examples of accessory cells include Fc receptor bearing accessory cells, PBMC dendritic cells, B cells, and monocytes. Other types of cells are contemplated.

Examples of exogenous growth factors include IL-2, IL-7 and IL-15, IL-17, IL-18 and IL-21 (and combinations of two or more of the aforementioned growth factors). Other types of growth factors are contemplated.

In certain embodiments, the exogenous growth factor comprises IL-2 and/or IL-7.

In certain embodiments, the method further comprises separating expanded T cells from the porous scaffold.

For example, depending upon the nature of the porous scaffold, the cells may be separated from the porous scaffold by separating liquid medium comprising cells from solid porous scaffold, sedimenting or centrifuging porous scaffold from cells in a liquid medium, or in the case where the porous scaffold forms part of a culture vessel, simply removing liquid comprising cells from the culture vessel. Other methods are contemplated.

Certain embodiments of the present disclosure comprise T cells expanded by a method as described herein.

Certain embodiments of the present disclosure provide a method of stimulating proliferation of a T cell, the method comprising exposing a T cell to a porous scaffold comprising one or more conjugated T cell stimulatory molecules to stimulate proliferation of the T cell.

T cells are as described herein. T cell stimulatory molecules are as described herein.

Porous scaffolds, and porous scaffolds comprising one or more conjugated T cell stimulatory molecules, are as described herein. Methods for assessing proliferation of T cells are known in the art.

In certain embodiments, the method comprises stimulating proliferation of a T cell sub-population.

In certain embodiments, the porous scaffold comprises a fibrous scaffold. In certain embodiments, the porous scaffold comprises an electrospun scaffold.

In certain embodiments, the porous scaffold is produced by melt electrospinning. In certain embodiments, the porous scaffold is produced by a method using melt electrospinning. In certain embodiments, the porous scaffold comprises a melt electrospun scaffold.

Certain embodiments of the present disclosure provide a composition comprising one or more T cells and a porous scaffold comprising one or more conjugated T cell stimulatory molecules.

T cells, porous scaffold, and porous scaffolds comprising one or more conjugated T cell stimulatory molecules, are as described herein.

In certain embodiments, the porous scaffold comprises a fibrous scaffold. In certain embodiments, the porous scaffold comprises an electrospun scaffold.

Certain embodiments of the present disclosure provide a complex comprising a T cell bound to a porous scaffold comprising one or more conjugated T cell stimulatory molecules.

T cells, porous scaffolds, and porous scaffolds comprising one or more conjugated T cell stimulatory molecules, are as described herein.

In certain embodiments, the porous scaffold comprises a fibrous scaffold. In certain embodiments, the porous scaffold comprises an electrospun scaffold.

Certain embodiments of the present disclosure provide a kit for performing a method as described herein.

In certain embodiments, the kit is used for activating and/or expanding T cells as described herein.

Certain embodiments of the present disclosure provide a kit for activating and/or expanding T cells, the kit comprising a porous scaffold comprising one or more conjugated T cell stimulatory molecules.

The kits may include one or more reagents as described herein, and/or instructions for performing a method as described herein.

For example, a kit may comprise the porous scaffold with one or more T cell stimulatory molecules, and one or more of buffers, additives, stabilisers, diluents, culture media, agents, growth factors, consumables, tissue culture vessels, plates, flasks, and instructions.

In certain embodiments, the porous scaffold comprises a fibrous scaffold. In certain embodiments, the porous scaffold comprises an electrospun scaffold.

Certain embodiments of the present disclosure provide a method of producing a porous scaffold with one or more conjugated T cell stimulatory molecules as described herein.

Certain embodiments of the present disclosure provide a method of producing a porous scaffold for activating and/or expanding a T cell, the method comprising conjugating one or more T cell stimulatory molecules to a porous scaffold.

Porous scaffolds are as described herein. In certain embodiments, the porous scaffold comprises electrospun fibres. In certain embodiments, the porous scaffold comprises electrospun fibres.

In certain embodiments, the porous scaffold comprises a fibrous scaffold. In certain embodiments, the porous scaffold comprises an electrospun scaffold.

T cell stimulatory molecules are as described herein.

In certain embodiments, the method comprises functionalisation of the porous scaffold. Methods for functionalisation of substrates and scaffolds are known in the art.

In certain embodiments, the functionalisation of the porous scaffold comprises plasma polymerisation. Methods for plasma polymerisation are known in the art.

In certain embodiments, the functionalisation of the porous scaffold comprises plasma polymerisation with a suitable monomer such as allyl glycidyl ether, allylamine, heptylamine, acrylic acid, 1,7 octadiene and ethanol.

Methods for conjugating one or more T cell stimulatory molecules to a porous scaffold are described herein.

In certain embodiments, the conjugating of the one or more T cell stimulatory molecules to a porous scaffold comprises exposing/incubating the functionalised porous scaffold with the one or more stimulatory molecules to covalently bond the molecules to the functionalised scaffold.

Certain embodiments of the present disclosure provide a porous scaffold with one or more conjugated T cell stimulatory molecules produced by a method as described herein.

In certain embodiments, the porous scaffold with one or more conjugated T cell stimulatory molecules is part of a product for activating, expanding, and/or culturing T cells.

In certain embodiments, the porous scaffold with one or more conjugated T cell stimulatory molecules is a component of a kit.

Certain embodiments of the present disclosure provide a product comprising a porous scaffold comprising one or more conjugated T cell stimulatory molecules.

In certain embodiments, the product is a cell culture vessel, such as a tissue culture flask, a tissue culture plate, a tissue culture tube, or a tissue culture dish. Examples include a Wave bag (GE Healthcare Life Sciences) or a gas-permeable rapid expansion flask (G-Rex flask—Wilson Wolf Manufacturing). Other types of products are contemplated. Methods for incorporating porous scaffolds into products are known in the art.

For example, a porous scaffold as described herein may be attached to, fixed to, tethered to, or incorporated in, a culture vessel, and one or more T cell stimulatory molecules conjugated to the porous scaffold. Activation and/or expansion of the T cells may then be performed in the culture vessel and the cells after activation and/or expansion removed by removal of the tissue culture medium.

Certain exemplary embodiments are illustrated by some of the following examples. It is to be understood that the following description is for the purpose of describing particular embodiments only and is not intended to be limiting with respect to the above description.

### Example 1 Electrospun Porous Scaffolds Coated with Stimulating Antibodies Provide a Substrate for Expansion of Lymphocytes Including CD4⁺, CD8⁺ and NK T Cells

(i) Scaffold Manufacture Via Melt Electrospinning

The electrospinning process and apparatus used were as described in Brown et al. (2014) Materials Science and Engineering: C, 2014, 45, 698-708, with some modifications as described.

For the electrospinning apparatus, the apparatus utilised the following:

(a) A Wood/Perspex safety enclosure equipped with a magnetic interlock to switch off high voltage while the enclosure door remains open was used.

(b) A printing head and associated reservoirs and pumps, able to produce and maintain a controlled flow of molten polymer was used. A 5 mL Hamilton glass syringe loaded with medical grade poly(ε-caprolactone) was placed into a heated custom-made PEEK printing head. A blunt needle (23G) was connected to the syringe, and the syringe was connected to the pump through a wooden adapter to feed the molten polymer through the needle. A coil heater with temperature controller was used to maintain polymer at constant temperature.

(c) A movable stage and associated controllers were used: high Speed CNC packing containing XY motors, drivers and interface card which connected the stage control box to a PC through a parallel port. Mach3 software was used to control XY movements. A polished stainless steel metal collector plate (200×200 mm²) was placed on the movable stage.

(d) Voltage differential control to the printing head, consisting of an in-house designed high voltage control device, digital multimeter and high voltage probe. The needle was connected to high voltage while the collector plate was grounded using high voltage wires.

The workspace was cleaned by applying 70% (vol/vol) ethanol. A syringe was filled with medical grade poly (ε-caprolactone) (mPCL) (Corbion Purac, solid pelletised PURASORB® PC12) to approximately ⅔ and placed into the electrospinning head. Electrospinning head was heated up to 100° C. for at least 150 min to ensure homogeneously molten polymer, then the syringe was connected to the pump. Polymer feeding rate was set at 10 to 25 μl/h and a 15 mm-25 mm distance between needle tip and collector established. The enclosure was closed and a high voltage differential between 1 1 kV and 13 kV was applied. Molten mPCL flow was monitored until the jet was stable. Fibre stability was achieved by adjusting the temperature, voltage and flow rate manually. At this point, computer control of the stage is initiated. Specific G-codes to translate CAD design of scaffold to the Mach3 CNC control software were written to fabricate 150 mm×150 mm scaffolds (pore sizes: 200 μm, 500 lam and 1000 μm, laydown pattern 0/90 degrees). At the end of the fabrication run, the voltage differential was reduced and the scaffold removed from the collector plate. Scaffolds were cut to size using a laser cutter, using a predefined CAD path. Post-processing, scaffolds were handled with tweezers, placed into shipping containers and the container sealed with parafilm.

(ii) Scaffold Functionalisation

Scaffolds were first plasma polymerised to produce an epoxy functionalised surface. The custom built plasma rig and its operation have been previously described, as described in Thierry B. et al. (2008) Langmuir, 24, 10187-10195, Dai L. et al. (1997) The Journal of Physical Chemistry B, 101, 9548-9554, and McLean K M et al. (2000) Colloids Surf B Biointerfaces, 18, 221-234.

Allyl glycidyl ether (>99%) was obtained from Sigma-Aldrich. The plasma deposition protocol utilised 2 steps of plasma polymerisation, as follows:

Step 1: constant wave plasma polymerisation (cw) for 1 min;

Step 2: pulsed plasma polymerisation (DC, i.e. duty cycle 1 ms/20 ms) for 2 min.

The pressure of the allyl glycidyl ether monomer during the treatment was at 0.2 Torr; plasma power was 25W.

After plasma polymerization, scaffolds were transferred to 48 well tissue culture treated plates for antibody conjugation. Scaffolds were incubated in a 320 μl volume of solution (40 μg/μl anti-human CD3 (Affymetrix eBioscence, Cat. No: 16-0039-81, functional grade purified) and 40 μg/μl anti-human CD28 (BD Pharmingen Cat. No: 555725, NA/LE CD28.2 functional grade purified)) in Dulbecco's phosphate buffered saline overnight. Antibody conjugated scaffolds were rinsed in copious Dulbecco's phosphate buffered saline before use.

CD4⁺ T Cell Isolation

Fresh buffy coats were obtained from the Australian Red Cross with approval for research use. Enrichment of CD4⁺ lymphocytes was achieved by RosetteSep (Stem Cell Technologies) negative selection, as per the manufacturer's protocol. Buffy coat cells were transferred into an appropriately sized flask and RosetteSep human CD4⁺ T Cell enrichment cocktail (Stem Cell Technologies, Cat. No: 15062) added at 1 ml per 50 ml of buffy coat. The mixture was incubated on an orbital mixer for 20 min at room temperature, at approx. 300 rpm, then diluted with Dulbecco's phosphate buffered saline (PBS, GE Healthcare Life Sciences, Cat. No: SH30028.02)+2% fetal bovine serum (FBS, Gibco, Cat. No: 10099) at a 1:2 ratio and mixed gently. 15 mL of Ficoll-Paque (density 1.077±0.001 g/ml (+20° C.), GE Healthcare Bio-Sciences AB, Cat. No: 17-1440-03) was placed in a 50 ml Falcon tube, and 35 ml of the diluted blood slowly layered over the Ficoll-Paque to avoid mixing. Density sedimentation was used to isolate the CD4⁺ cell population by centrifuging the RosetteSep treated buffy coat through the Ficoll-Paque for 25 min at 400 g at RT, with no deceleration brake. Enriched CD4⁺ cells were removed from the plasma interphase into a new tube and washed twice with PBS+2% FBS. Isolated CD4⁺ T cells were maintained in X-Vivo™15 complete medium with gentamicin and phenol red (Lonza, Cat. No: 04-418Q) supplemented with 5% human serum (Sigma, Cat. No: H4522-100), 20 nM HEPES (Gibco, cat. no: 15630-080) and 2 mM L-glutamine (Sigma, cat. no: G7513) plus IL-2 (500 U/ml, Novartis Vaccines and Diagnostics) prior to use. CD4⁺ lymphocytes were seeded onto scaffolds within 72 h of isolation.

(iii) CD4⁺ T Cell Culture on Scaffolds

Before cell seeding, scaffolds were placed in individual wells of a 48 well tissue culture plate. Triplicate scaffolds were included for each scaffold spacing dimension. CD4⁺ lymphocytes were cultured on each scaffold, using a starting density of 1.25×10⁵ cells in a 0.5 mL volume for a period of 5 days. Cultures were left undisturbed (without medium exchange or addition) during the culture period. At the end of the culture, any adherent cells attached to the scaffold were removed by trituration and the cell suspension quantified by haemocytometer (Nielsen L K et al. (1991) Biotechnol. Prog., 7, 560-563).

Results

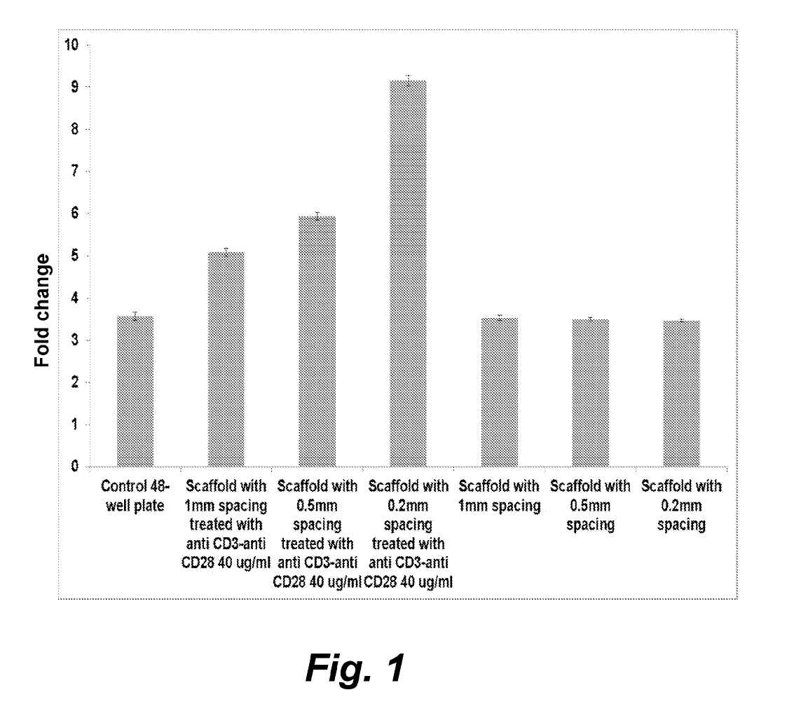

The results are shown in FIG. 1. The fibrous scaffolds produced by electrospinning and coated with co-stimulatory molecules (antibodies anti-CD3 and anti-CD28) provided a substrate that induced increased proliferation of the CD4⁺ lymphocytes, compared with the residual cell expansion that occurred in cultures containing uncoated scaffolds or no scaffolds (approx. 2 fold), demonstrating that robust cell expansion was dependent upon the presence of the co-stimulatory molecules on the scaffold.

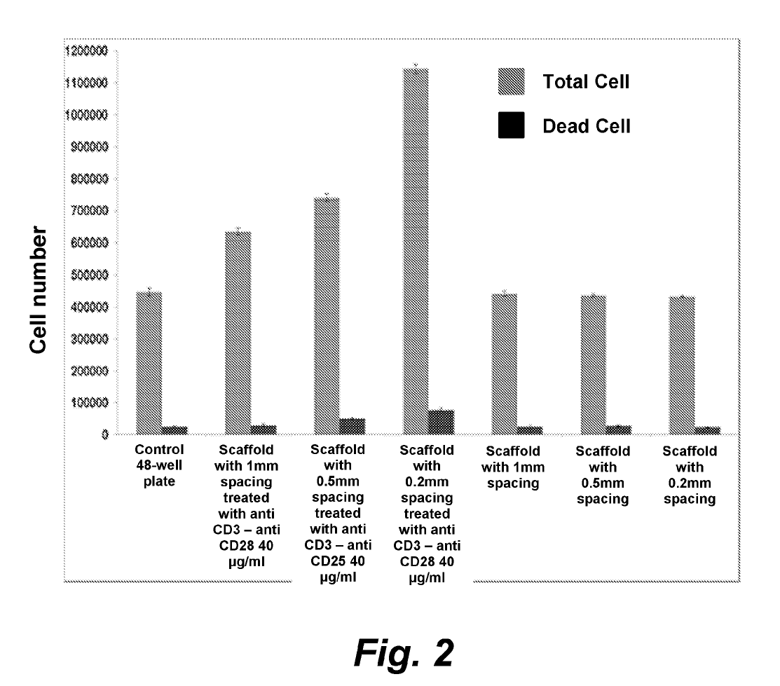

Further improvements in cell expansion (approx. 9 fold) over the five day culture period were achieved by decreasing fibre spacing, and improved cell expansion using antibody-conjugated mPCL scaffolds was obtained with a fibre spacing of 0.2 mm (FIG. 1). All antibody coated scaffolds tested produced cell expansion above the baseline level (2 fold). This data indicates that reducing the spacing between fibres improves cell expansion, at that in the experiments conducted, a spacing of 0.2 mm (200 μm) gave improved cell expansion. Cell viability at the end of the five day culture on the antibody coated 0.2 mm fibre spacing mPCL scaffold remained above 90% (FIG. 2), showing that the scaffold does not have toxic effects over the time tested.

### Example 2 Coating Antibody Concentration

The scaffolds, the conjugation of antibodies and the expansion of CD4⁺ lymphocytes were as described in Example 1, using fabrication of mPCL scaffolds with a 0.2 mm spacing and epoxy plasma functionalisation. The only changes to the protocol were as follows:

i) The addition of an extra condition—80 μg/mL anti CD3 antibody and 80 μg/mL anti CD28 antibody—during antibody coating of scaffolds. All antibody solutions were 1:1 anti-CD3: anti-CD28.

ii) Substitution of a non-tissue culture treated 48 well plate (Iwaki) for a tissue culture polystyrene plate as the vessel used for antibody conjugation of the scaffolds.

iii) Medium exchange at day 4 (80% medium exchange, 0.4 mL volume harvested from each culture, centrifuged 3 min, 150×g, 18° C., and cells recovered resuspended in fresh medium and returned to the culture).

Controls for the experiment included i) Dynal anti CD3/anti CD28 beads [cat#111.41D] at a 1:1 cell bead ratio and ii) epoxy plasma treated (without antibody) scaffold. Seeding density was 1.25×10⁶ per 0.5 mL culture.

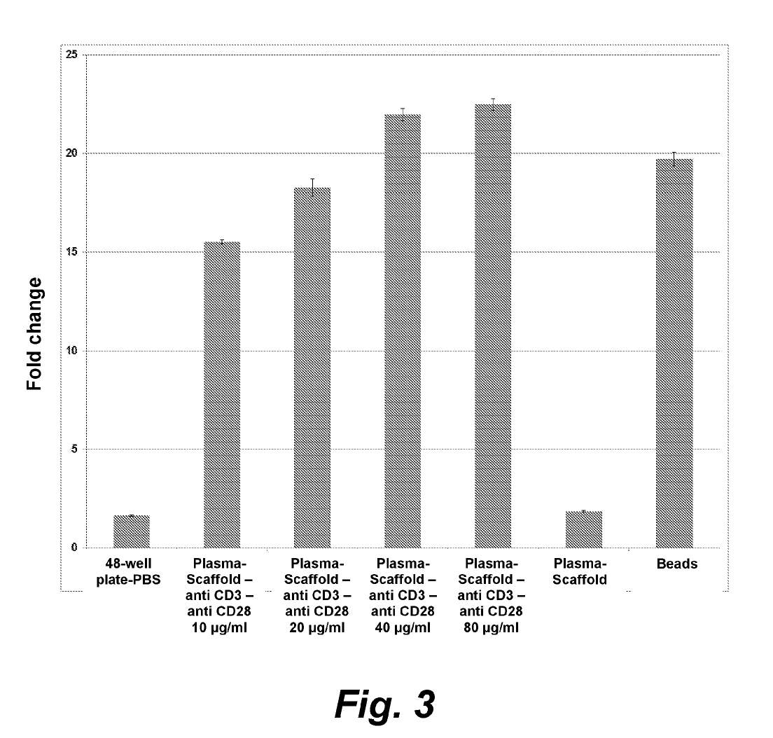

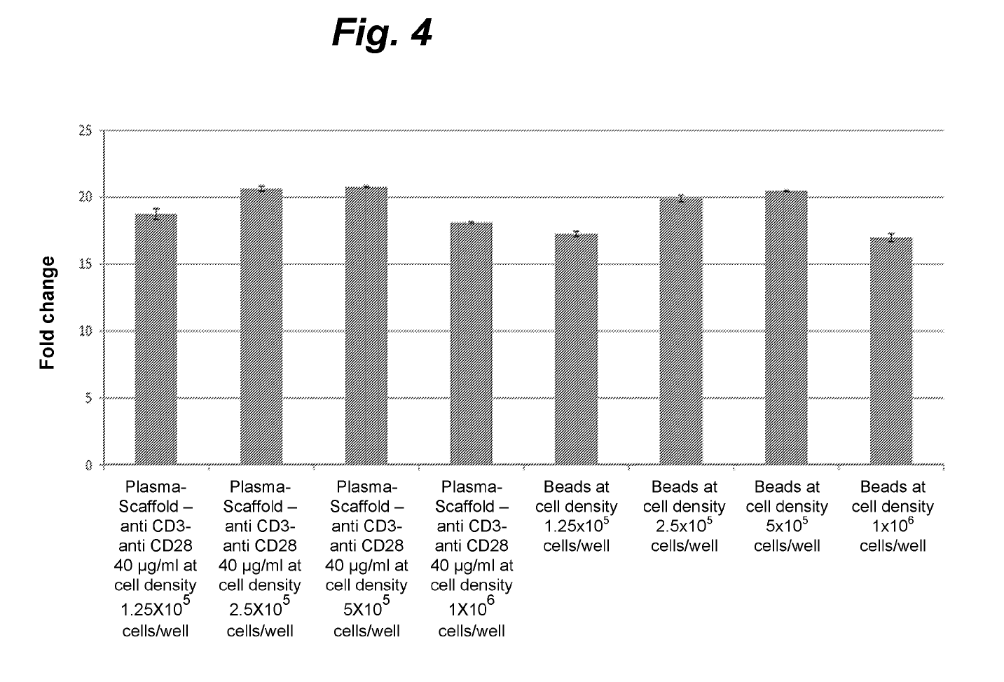

The data is shown in FIG. 3.

Robust cell expansion was observed even at the lowest concentration of each of the conjugated antibodies used (10 μg/ml). The fold expansion after 7 days for our previous condition showing desirable expansion under the conditions tested (40 μg/mL anti CD3 antibody and 40 μg/mL anti CD28 antibody) was 22 fold and was comparable to the expansion obtained with the Dynal beads. Increasing each antibody to 80 μg/mL resulted in a slight improvement over the results using 40 μg/ml of each of the antibodies. T-tests (one sided, unequal variance) showed a significant increase from 20 μg/mL to 40 μg/mL (P<0.002) but not from 40 μg mL to 80 μg/mL (p=0.15).

This data shows that the fibrous scaffold with conjugated antibodies is an effective system for expansion of CD4⁺ lymphocytes and comparable in performance to a bead conjugated system.

### Example 3 Expansion of Treg Cells on Scaffold at Different Cell Densities

Treg cells were isolated from CD4⁺ cells by fluorescence-based cell sorting (FACS). CD4⁺ cells isolated as described in Example 1, were labelled with antibodies to CD4 (anti-human CD4 APC-H7, cat#641398), CD25 (anti-human CD25-PECy7, cat#557741) and CD127. Treg cells were isolated based on CD4⁺, CD25⁺ and CD127-status using stringent gating, and Treg were used for the expansion experiment shown in FIG. 4.

The fold expansion of Treg cells after 7 days is shown in FIG. 4.

FIG. 4 demonstrates equivalent or better fold expansion of CD4⁺CD25⁺CD237⁻ Treg on the scaffold compared with beads. It was found that the cells showed good overall viability of Treg expanded on the scaffold, with a desirable cell density giving >80% viability.

### Example 4 Expansion of CD8⁺ Cells

Production and use of the porous scaffold with conjugated anti-CD3 antibody and anti-CD28 antibody were as described in Example 1, with the exception of RosetteSep purification, where a RosetteSep human CD8⁺ T Cell enrichment cocktail (Stem Cell Technologies, Cat. No: 15063) was used.

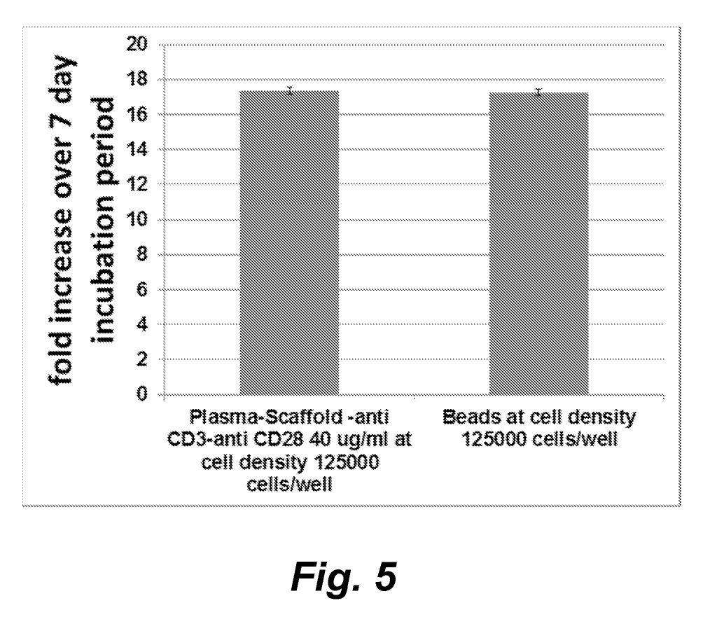

The expansion of the CD8⁺ cells is shown in FIG. 5. Expansion on scaffolds in comparison to beads is shown. The viability achieved in the end product for the two expansion matrices (scaffold and beads) is identical at 84.3%. The difference in cell yield between scaffold and beads was not statistically significant (p=0.69, T test, unequal variance).

### Example 5 Kits for Activating and/or Expanding T Cells

A kit for use for activating and/or expanding T cells may contain a porous a scaffold as described herein (for example either alone, lyophilised, in a preservative solution, or in combination with a suitable additive, such as being provided in a suitable buffer, such as PBS, and/or with serum albumin) and optionally one or more of the following components:
(i) Buffer such as PBS. The buffer may for example be supplemented with BSA/HSA (eg 0.1%) and EDTA (eg 2 mM, pH 7.4).
(ii) Culture medium, such as RPMI medium. The medium may for example be supplemented with human serum (eg 2%), L-Glutamine (eg 2 mM), and an antibiotic (eg penicillin/streptomycin—100 U).
(iii) Recombinant human IL-2 (eg 50 U IL-2/mL), or other T cell growth factor.
(iv) Recombinant human IL-7 (for expansion of CD8⁺ T cells).
(v) Tissue culture plates, tissue culture flasks, other forms of culture vessel, typically sterilised. The porous scaffold with one or more stimulatory molecules may also be integrated into such culture vessels.
(vi) Instructions for one or more of preparing cells for activation/expansion, preparing the porous scaffold, activating and expanding cells, restimulating cells, and isolating T cells.

One or more of the kit components of the kit may be packaged in sterile form.

### Example 6—Scaffold Supports Expansion of Human Regulatory T Cells (Treg Cells)

Methods

Human T Cell Isolation

Fresh buffy coats were obtained from the Australian Red Cross with ethics approval for research use (ARC13317). Enrichment of CD4⁺ lymphocytes was achieved by RosetteSep (Stem Cell Technologies, Australia) negative selection, as per the manufacturer's protocol. Buffy coat cells were transferred into a sterile tissue culture flask and RosetteSep human CD4⁺ T Cell enrichment cocktail added at 1 mL per 50 mL of buffy coat. The mixture was incubated with agitation using an orbital mixer for 20 min at room temperature, at approx. 300 rpm. Cells were then diluted with PBS (GE Healthcare Life Sciences, USA)+2% fetal bovine serum (FBS, Thermo Fisher Scientific, USA) at a 1:2 volume:volume ratio and mixed gently. Ficoll-Paque (15 mL, density 1.077±0.001 g/mL (+20° C.), GE Healthcare Life Sciences) was placed in a 50 mL Falcon tube, and 35 mL of the diluted blood slowly layered over the Ficoll-Paque to avoid mixing. Density sedimentation was used to isolate the CD4⁺ cell population by centrifuging the RosetteSep treated buffy coat through the Ficoll-Paque for 25 min at 400 g at room temperature, with no deceleration brake. Enriched CD4⁺ cells were removed from the plasma: ficoll interface into a new tube and washed twice with PBS+2% FBS. Isolated CD4⁺ T cells were maintained in X-Vivo 15 medium with gentamicin and phenol red (Lonza, Switzerland) completed with 5% human serum (Sigma-Aldrich), 20 nM HEPES (Thermo Fisher Scientific), 2 mM L-glutamine (Sigma-Aldrich), and IL-2 (500 U/mL, Novartis Vaccines and Diagnostics, USA) prior to use (complete X-Vivo 15 medium). CD4⁺ lymphocytes were seeded onto scaffolds within 72 h of isolation.

Treg Cell Isolation

For experiments using human regulatory T cells, the CD4⁺ population isolated as described above was further purified by fluorescent activated cell sorting. Cells were stained with antibodies to human CD4 (APC), CD25 (PECy7) and CD127 (FITC), all obtained from Beckton Dickinson following the manufacturer's protocol. A stringent CD25⁺CD127⁻ gating strategy was used to sort human regulatory T cells. Purity and phenotype were confirmed by FOXP3 staining before and after culture. FOXP3 staining was performed using an intracellular fixation and permeabilisation protocol as per the manufacturer's protocol (Anti-human FOXP3 Alexa Fluor 647, Beckton Dickinson). Cell sorting was performed using a FACSAria2 (Beckton Dickinson), and cell analysis was performed using a FACSCanto2 flow cytometer (Beckton Dickinson).

T Cell Expansion

T cell subsets isolated as described above were cultured in 48 well plates at 5×10⁵/mL in contact with the CD3/CD28 functionalised scaffolds in the presence of 500 U/mL human IL2, and cultures were maintained at 37° C., 5% CO₂ in a humidified cell culture incubator for 7 days. For comparison, donor-matched cells were also cultured at the same starting density (10⁶/mL) with 1:1 Dynabeads® Human T-Activator CD3/CD28 beads (Thermo Fisher Scientific), to establish relative stimulation compared with the bead-based antibody display.

Mixed Lymphocyte Reaction

The mixed lymphocyte reaction was performed as outlined in Hill et al³⁸. Effector CD4⁺CD25⁻ cells were isolated as described above and labelled with 1 μM carboxyfluorescein succinimidyl ester (CFSE, Invitrogen) in PBS for 10 min at 37° C. The reaction was quenched by addition of 5×volume of ice-cold complete X-Vivo 15 medium. CFSE labelled effector cells (2×10⁴ per well) were incubated with 1×10⁵ irradiated (30 Gy) unmatched PBMCs in U-bottom 96 well plates. Treg cells were added at Teffector:Treg ratios of 1:1, 2:1, 4:1, 8:1, 16:1, and 32:1 in the presence of 100 ng/mL anti-CD3 (OKT3) monoclonal antibody (eBioscience, USA) in a final volume of 200 pt complete X-Vivo 15 per well in a 96-well U-bottom plate. Co-cultures were harvested after 5 d of incubation and the proliferation of the effector population was visualised by the dilution of CFSE fluorescence with cell division on a BD FACSAriaII flow cytometer.

QPCR

Total RNA was extracted from all cell populations using the RNeasy kit (Qiagen, Germany) and subsequently converted to cDNA using the Quantitect Reverse Transcription kit (Qiagen). Semi-Quantitative RT-PCR was performed using the KAPA SYBR Fast Universal qPCR kit (KAPA Biosystems, USA) in triplicate. PCR reactions were performed on a Corbett real time PCR machine (Rotorgene 6000, Qiagen). Results from three independent experiments were analysed using Rotor-Gene 6000 software and normalised to the expression of reference transcript ribosomal protein L13a (RPL13a). The sequences of RT PCR primers were as follows: FOXP3 forward -5′-ATGGCCCAGCGGATGAG-3′ (SEQ ID NO. 1), and reverse 5′-GAAACAGCACATTCCCAGAGTTC-3′ (SEQ ID NO. 2); CTLA4 forward -5′-CATGATGGGGAATGAGTTGACC-3′ (SEQ ID NO. 3), and reverse 5′-TCAGTCCTTGGATAGTGAGGTTC-3′(SEQ ID NO. 4), RPL13a forward 5′-CGAGGTTGGCTGGAAGTACC-3′ (SEQ ID NO. 5) and reverse 5′-CTTCTCGGCCTGTTTCCGTAG-3′ (SEQ ID NO. 6). Estimates of log 2 fold-change were obtained using the ΔΔCt method normalising to the housekeeper RPL13a.

Results

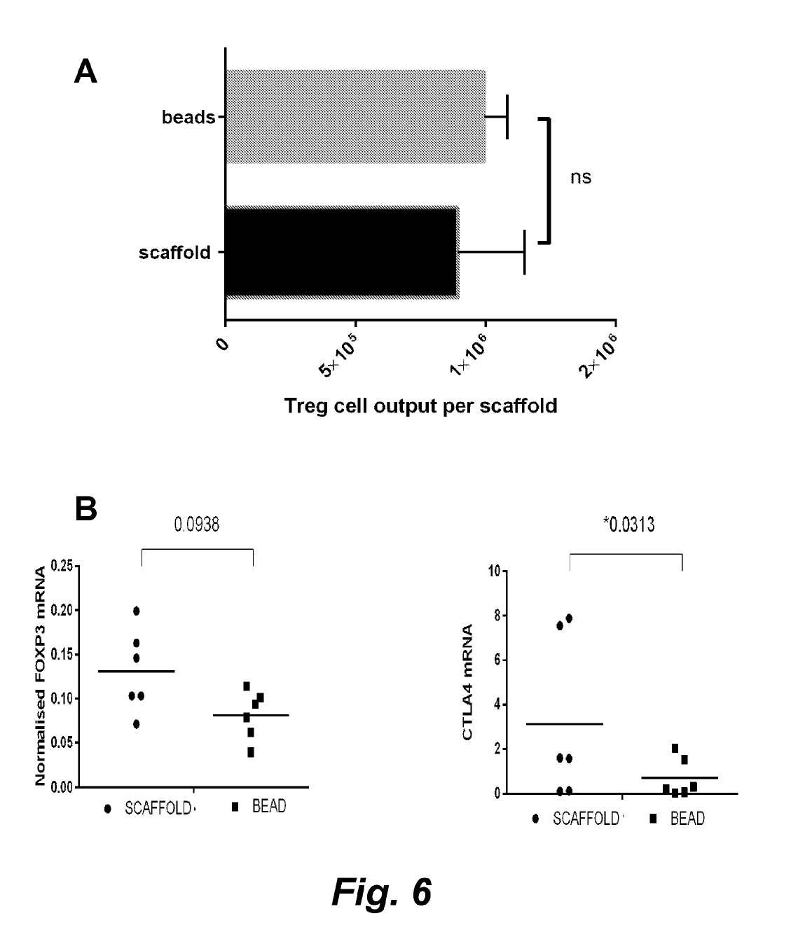

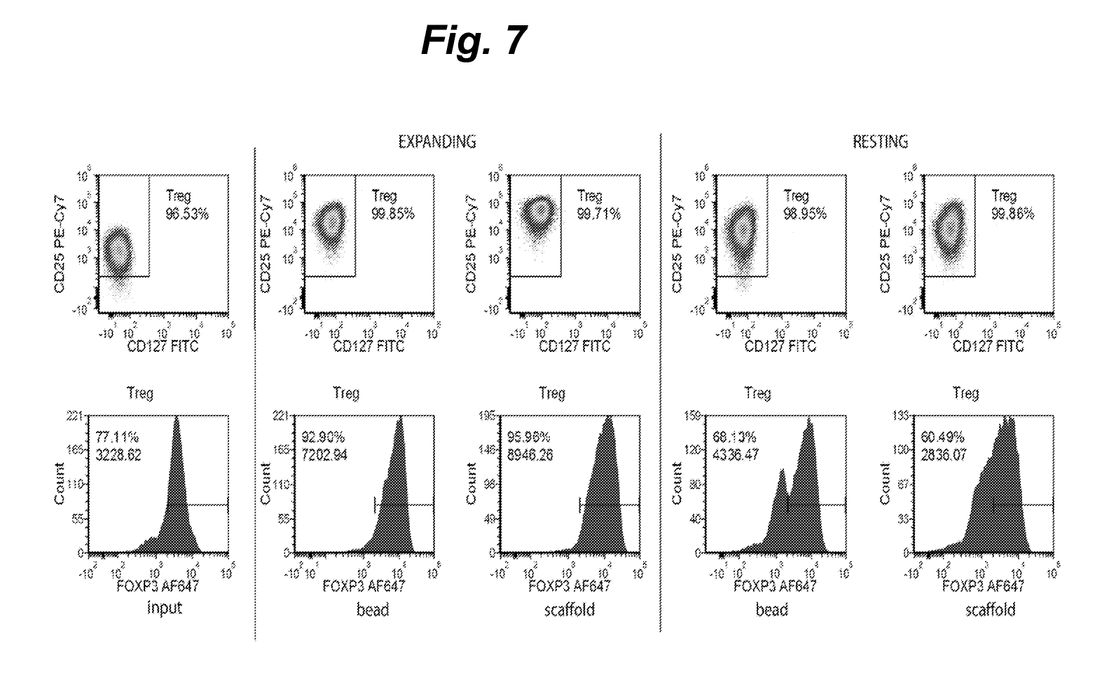

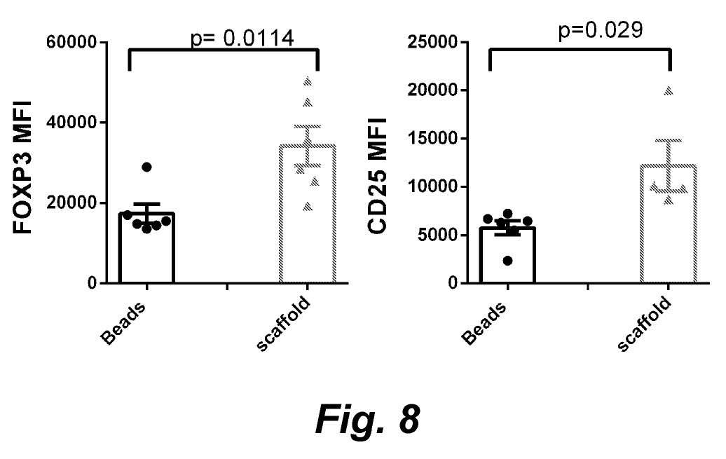

We tested the ability of the scaffold to support the expansion of human Treg cells. Treg cells were isolated from total human CD4⁺ pools by fluorescence-based cell sorting. A stringent gating strategy isolating CD4⁺CD25⁺CD127^{dim} cells was used and we routinely recovered 0.5-4×10⁶ Treg cells from 1-2×10⁸ peripheral blood mononuclear cells, with post sort purity of >85%. A key feature of human Treg cells is the expression of the transcription factor FOXP3, and intracellular staining for expression of FOXP3 during and after expansion is a critical quality control step. Our input Treg cell populations are routinely >95% FOXP3⁺ as determined by intracellular antibody staining. As can be seen in FIG. 6A, Treg cells responded in vitro to culture on the 3D scaffold, and proliferated at a similar rate to Treg exposed to CD3/CD28 beads using donor matched input cells. The Treg cells expanded in this system retained high purity based on bright staining for CD25. Both CD25 and FOXP3 mean fluorescence intensity (protein expression levels) at harvest was significantly higher and tighter from the 3D scaffold compared with the beads (FIG. 7). Of note, the output Treg cells from the expansion on the 3D scaffold retained robust FOXP3 expression (97% post expansion, 94% after 5 days rest) (FIG. 7), and also expressed elevated FOXP3, CTLA4, and CD25 at the RNA level (FIG. 6B, FIG. 8), consistent with a stable Treg cell phenotype. Critically, after 5 days rest post-expansion on the 3D scaffold, only a small number of CD25^{dim} or CD25⁻ cells were detected. In contrast, the bead culture contained significant numbers of CD25⁻ cells post-rest, which are a potential contaminant T effector population that can expand in vitro under the same conditions. In addition the CD25⁺ gate contained ˜85% FOXP3⁺ cells (FIG. 7).

The MFI of FOXP3 was at least 1.5 times higher for expansion on the scaffold as compared to beads, and this was also reflected in the level FOXP3 mRNA which was two times higher for the scaffold than beads.

In the case of the MFI for CD25, greater than 50% of the scaffold expanded cells were CD25⁺⁺, as compared with bead expanded cells, where less than 50% were CD25 bright.

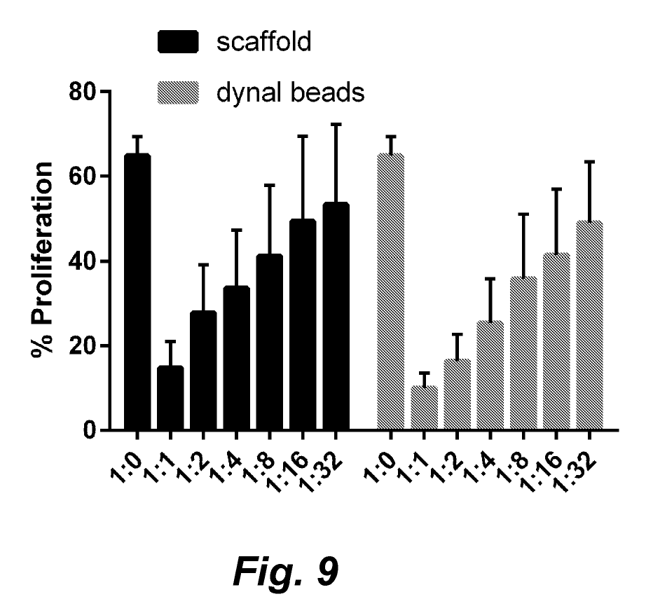

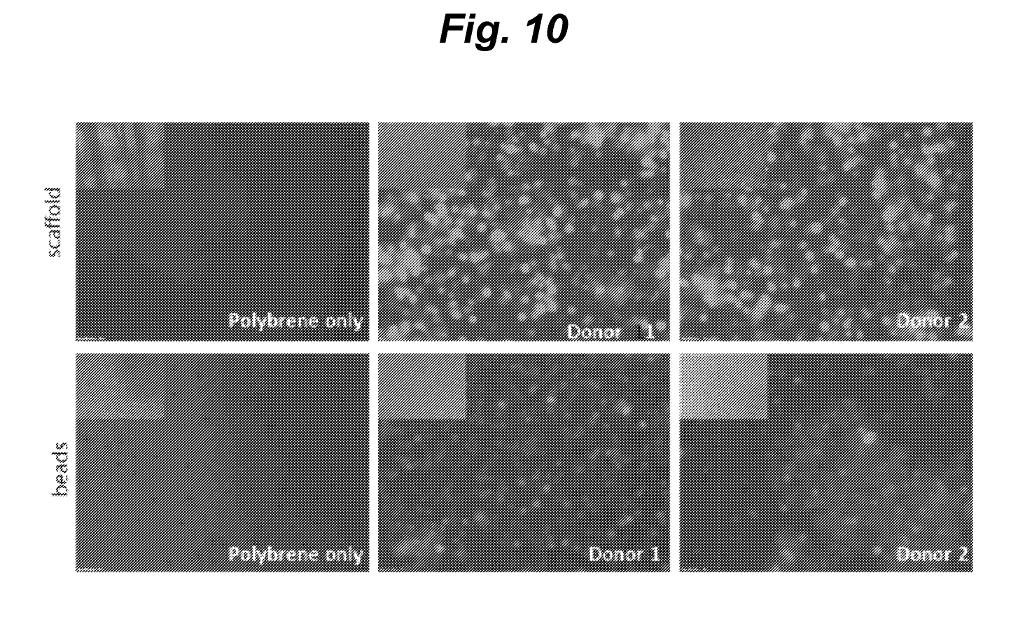

Finally, the scaffold expanded Treg cells also showed increased suppressor function in the mixed lymphocyte suppressor assay (MLR), which is the gold standard for a functional Treg cell population. A Treg cell dose-dependent titration of suppression of proliferation was observed from 3D scaffold-expanded donor-matched Treg compared with Bead expanded Treg cells (FIG. 9). Expanded Treg cells showed a 20% or greater suppression of proliferation at 1:8 than bead expanded cells at 1:8.

Conclusion

Treg cells can be successfully expanded on the scaffold, to produce a similar yield as produced by Dynal beads over a 7 day culture period.

Treg cell phenotype at the end of the scaffold-based culture indicates higher expression of Treg cell markers CD127⁺CD25⁺ than achieved with beads. Treg cell identity is confirmed by CTLA4 and FOXP3 expression. Treg cell phenotype is retained after 5 days rest culture, illustrating that phenotype is stable and not an artefact of activation.

Treg retain functionality after expansion on scaffolds.

### Example 7—Scaffold Enables Lentiviral Gene Delivery to Human CD4⁺ T Cells Methods

Human T Cell Isolation

Fresh buffy coats were obtained from the Australian Red Cross with ethics approval for research use (ARC13317). Enrichment of CD4⁺ lymphocytes was achieved by RosetteSep (Stem Cell Technologies, Australia) negative selection, as per the manufacturer's protocol. Buffy coat cells were transferred into a sterile tissue culture flask and RosetteSep human CD4⁺ T Cell enrichment cocktail added at 1 mL per 50 mL of buffy coat. The mixture was incubated with agitation using an orbital mixer for 20 min at room temperature, at approx. 300 rpm. Cells were then diluted with PBS (GE Healthcare Life Sciences, USA)+2% fetal bovine serum (FBS, Thermo Fisher Scientific, USA) at a 1:2 volume:volume ratio and mixed gently. Ficoll-Paque (15 mL, density 1.077±0.001 g/mL (+20° C.), GE Healthcare Life Sciences) was placed in a 50 mL Falcon tube, and 35 mL of the diluted blood slowly layered over the Ficoll-Paque to avoid mixing. Density sedimentation was used to isolate the CD4⁺ cell population by centrifuging the RosetteSep treated buffy coat through the Ficoll-Paque for 25 min at 400 g at room temperature, with no deceleration brake. Enriched CD4⁺ cells were removed from the plasma: ficoll interface into a new tube and washed twice with PBS+2% FBS. Isolated CD4⁺ T cells were maintained in X-Vivo 15 medium with gentamicin and phenol red (Lonza, Switzerland) completed with 5% human serum (Sigma-Aldrich), 20 nM HEPES (Thermo Fisher Scientific), 2 mM L-glutamine (Sigma-Aldrich), and IL-2 (500 U/mL, Novartis Vaccines and Diagnostics, USA) prior to use (complete X-Vivo 15 medium). CD4⁺ lymphocytes were seeded onto scaffolds within 72 h of isolation.

T Cell Expansion

T cell subsets isolated as described above were cultured in 48 well plates at 5×10⁵/mL in contact with the CD3/CD28 functionalised scaffolds in the presence of 500 U/mL human IL2 for 4 d prior to transduction, and cultures were maintained at 37° C., 5% CO₂ in a humidified cell culture incubator. For comparison, donor-matched cells were also cultured at the same starting density (10⁶/mL) with 1:1 Dynabeads® Human T-Activator CD3/CD28 beads (Thermo Fisher Scientific), to establish relative stimulation compared with the bead-based antibody display.

Lentivirus Transduction

High titre GFP expressing lentiviral stocks (LVGFP) were prepared as previously described using third generation self-inactivating lentiviral vectors pseudo typed with VSG-G. Virus was concentrated by ultracentrifugation prior to use. Primary human CD4⁺ T cells isolated and cultured as described above were quantified and 5×10⁵ cells transduced with LVGFP at a Multiplicity of Infection (MOI) of 20 overnight in the presence of 20 ng/ml Polybrene. Control cultures with polybrene only were performed in parallel. The transduction was repeated the following day, and then cells washed and re-plated in to X-vivo 15 with supplements and incubated for 72 h. Transduction efficiency was determined by FACS analysis of GFP expression % and mean fluorescent intensity.

Results

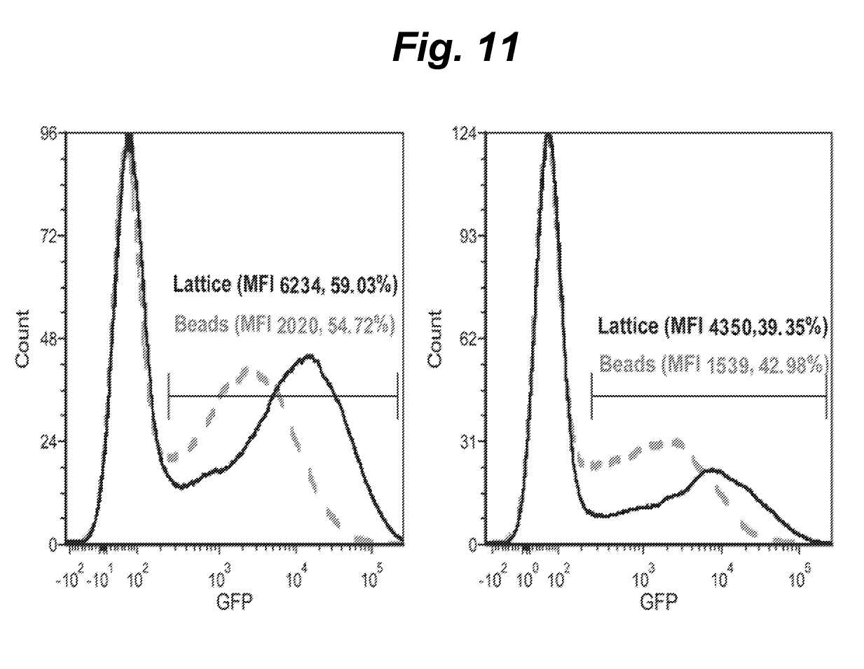

As a further proof of utility for expansion of clinically relevant cell types, we tested the ability of the 3D scaffold to facilitate lentiviral gene delivery. CD4⁺ T cells were efficiently transduced by lentiviral vectors expressing GFP (FIG. 10), and the scaffold activated cells showed higher lentiviral gene expression per cell based on mean fluorescent intensity (FIG. 11).

Conclusions

The scaffold is compatible with lentivirus transduction of T lymphocytes

Mean expression of exogenous gene is higher when transduction is performed on scaffolds compared to beads.

### Example 8—Scaffolds Support Expansion of Human CD8⁺ T Cells

Methods

Human T Cell Isolation

Fresh buffy coats were obtained from the Australian Red Cross with ethics approval for research use (ARC13317). Enrichment of CD4⁺ lymphocytes was achieved by RosetteSep (Stem Cell Technologies, Australia) negative selection, as per the manufacturer's protocol. Buffy coat cells were transferred into a sterile tissue culture flask and RosetteSep human CD8⁺ T Cell enrichment cocktail added at 1 mL per 50 mL of buffy coat. The mixture was incubated with agitation using an orbital mixer for 20 min at room temperature, at approx. 300 rpm. Cells were then diluted with PBS (GE Healthcare Life Sciences, USA)+2% fetal bovine serum (FBS, Thermo Fisher Scientific, USA) at a 1:2 volume:volume ratio and mixed gently. Ficoll-Paque (15 mL, density 1.077±0.001 g/mL (+20° C.), GE Healthcare Life Sciences) was placed in a 50 mL Falcon tube, and 35 mL of the diluted blood slowly layered over the Ficoll-Paque to avoid mixing. Density sedimentation was used to isolate the CD8⁺ cell population by centrifuging the RosetteSep treated buffy coat through the Ficoll-Paque for 25 min at 400 g at room temperature, with no deceleration brake. Enriched CD8⁺ cells were removed from the plasma: ficoll interface into a new tube and washed twice with PBS+2% FBS. Isolated CD8⁺ T cells were maintained in X-Vivo 15 medium with gentamicin and phenol red (Lonza, Switzerland) completed with 5% human serum (Sigma-Aldrich), 20 nM HEPES (Thermo Fisher Scientific), 2 mM L-glutamine (Sigma-Aldrich), and IL-2 (500 U/mL, Novartis Vaccines and Diagnostics, USA) prior to use (complete X-Vivo 15 medium). CD8⁺ lymphocytes were seeded onto scaffolds within 72 h of isolation

T Cell Expansion

T cell subsets isolated as described above were cultured in 48 well plates at 5×10⁵/mL in contact with the CD3/CD28 functionalised scaffolds in the presence of 500 U/mL human IL2 for 7 days, and cultures were maintained at 37° C., 5% CO₂ in a humidified cell culture incubator. For comparison, donor-matched cells were also cultured at the same starting density (10⁶/mL) with 1:1 Dynabeads® Human T-Activator CD3/CD28 beads (Thermo Fisher Scientific), to establish relative stimulation compared with the bead-based antibody display.

Results

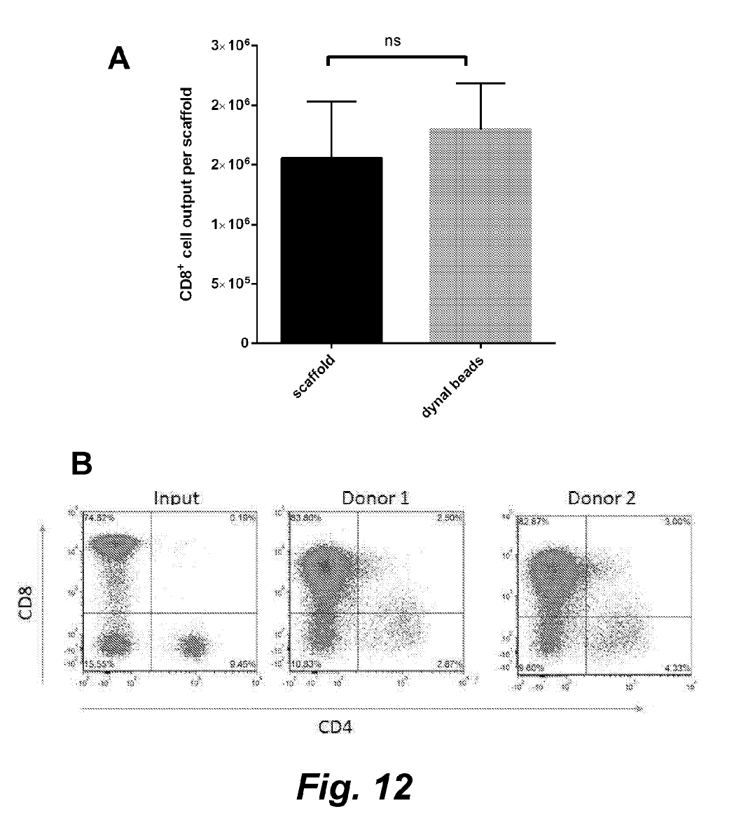

The utility of the 3D scaffold to enable expansion of other clinically relevant populations is further demonstrated by stimulation of human CD8⁺ T cells isolated using a MACS-based protocol. Robust proliferation of CD8⁺ T cells on the 3D scaffold and approximately 10 fold expansion were achieved in 5 days (FIG. 12A). The purity and surface staining characteristics of the CD8⁺ T cells are retained after 5 days in culture (FIG. 12B).

Conclusions

CD8⁺ cells can be successfully expanded on the scaffold. Phenotype is maintained during expansion.

### Example 9—Scaffold Architecture and Scaffold Coating Conditions for Expansion of T Lymphocytes

Aim: to determine the characteristics of scaffold architecture (fibre diameter, fibre spacing, and number of layers in scaffold) and scaffold coating conditions for expansion of T lymphocytes.

Methods

Scaffold Fabrication

Highly-ordered cell culture scaffolds were manufactured using a custom-made melt electrospinning writing (MEW) device. The spinning process and printer used as described in Example 1, with some modifications as described below. Briefly, a 5 mL glass syringe (Gastight® Cat. No. 1005, Hamilton Co., USA) was loaded with medical grade polycaprolactone (mPCL) pellets (Purasorb® PC12, Purac Biomaterials, The Netherlands). The polymer was heated up at temperatures between 90-100° C. by a coil heater with temperature controller. The syringe was connected to a programmable syringe pump (AL-1000, World Precision Instruments Inc., USA) via a wooden adapter to extrude the molten polymer through a 23G Luer lock metal needle at a constant flow rate of 10 μm/h. A high voltage between 10.5 and 12.5 kV was applied to the needle (DX250R, EMCO High Voltage Co., USA) and a grounded stainless steel collector was maintained at a distance of 20 mm to the needle. When the extruded melted polymer flew through the spinneret, an electrostatically drawn jet was accelerated toward the grounded collector. A motorised XY positioning slide (Velmex Inc., USA) driven by a CNC controller software (Mach3, Newfangled Solutions, USA) was used to displace the collector with programmable patterns to collect scaffolds of defined fibre spacing and orientation: 200 μm, 500 μm and 1000 μm fibre spacing and a lay-down pattern of 0°/90°. Scaffolds were cut to size using a laser-cutting machine (ILS12.75, Universal Laser Systems, Inc. USA).

Disorganised scaffolds were electrospun on the same apparatus and created with the same porosity as the 200 micron direct write scaffolds.

Surface Functionalisation

Scaffolds were coated with a plasma polymer to produce an epoxy-functionalised surface. Allyl glycidyl ether (>99%) was obtained from Sigma-Aldrich (USA). The plasma deposition protocol utilised 2 steps of plasma polymerisation, as follows: Step 1: constant wave plasma polymerisation (cw) for 1 min; Step 2: pulsed plasma polymerisation (DC, i.e. duty cycle 1 ms/20 ms) for 2 min. The pressure of the allyl glycidyl ether monomer during the treatment was at 0.2 Torr; plasma power was 25 W.

After plasma polymerisation, scaffolds were transferred to 48 well tissue culture-treated plates for antibody conjugation. Scaffolds were incubated in a solution containing a 1:1 ratio of 40 μg/μL anti-human CD3 functional grade purified antibody (Affymetrix eBioscience, USA) Cat. No: 16-0039-81, functional grade purified) and 40 μg/μL anti-human CD28 functional grade purified antibody (Becton Dickinson, USA) Cat. No: 555725, NA/LE CD28.2 functional grade purified)) in Dulbecco's phosphate buffered saline (PBS, Sigma-Aldrich) at 4° C. overnight. Scaffolds were produced using solutions containing 10, 20, 40 and 80 μg/mL of each antibody. Antibody-conjugated scaffolds were rinsed in copious PBS before use.

Human T Cell Isolation

Fresh buffy coats were obtained from the Australian Red Cross with ethics approval for research use (ARC13317). Enrichment of CD4⁺ lymphocytes was achieved by RosetteSep (Stem Cell Technologies, Australia) negative selection, as per the manufacturer's protocol. Buffy coat cells were transferred into a sterile tissue culture flask and RosetteSep human CD4⁺ T Cell enrichment cocktail added at 1 mL per 50 mL of buffy coat. The mixture was incubated with agitation using an orbital mixer for 20 min at room temperature, at approx. 300 rpm. Cells were then diluted with PBS (GE Healthcare Life Sciences, USA)+2% fetal bovine serum (FBS, Thermo Fisher Scientific, USA) at a 1:2 volume:volume ratio and mixed gently. Ficoll-Paque (15 mL, density 1.077±0.001 g/mL (+20° C.), GE Healthcare Life Sciences) was placed in a 50 mL Falcon tube, and 35 mL of the diluted blood slowly layered over the Ficoll-Paque to avoid mixing. Density sedimentation was used to isolate the CD4+ cell population by centrifuging the RosetteSep treated buffy coat through the Ficoll-Paque for 25 min at 400 g at room temperature, with no deceleration brake. Enriched CD4⁺ cells were removed from the plasma: ficoll interface into a new tube and washed twice with PBS+2% FBS. Isolated CD4⁺ T cells were maintained in X-Vivo 15 medium with gentamicin and phenol red (Lonza, Switzerland) completed with 5% human serum (Sigma-Aldrich), 20 nM HEPES (Thermo Fisher Scientific), 2 mM L-glutamine (Sigma-Aldrich), and IL-2 (500 U/mL, Novartis Vaccines and Diagnostics, USA) prior to use (complete X-Vivo 15 medium). CD4⁺ lymphocytes were seeded onto scaffolds within 72 h of isolation.

T Cell Expansion

T cell subsets isolated as described above were cultured in 48 well plates at 5×10⁵/mL in contact with the CD3/CD28 functionalised scaffolds in the presence of 500 U/mL human IL2 for 7 d, unless otherwise stated, and cultures were maintained at 37° C., 5% CO₂ in a humidified cell culture incubator. For comparison, donor-matched cells were also cultured at the same starting density (10⁶/mL) with 1:1 Dynabeads® Human T-Activator CD3/CD28 beads (Thermo Fisher Scientific), to establish relative stimulation compared with the bead-based antibody display.

Results

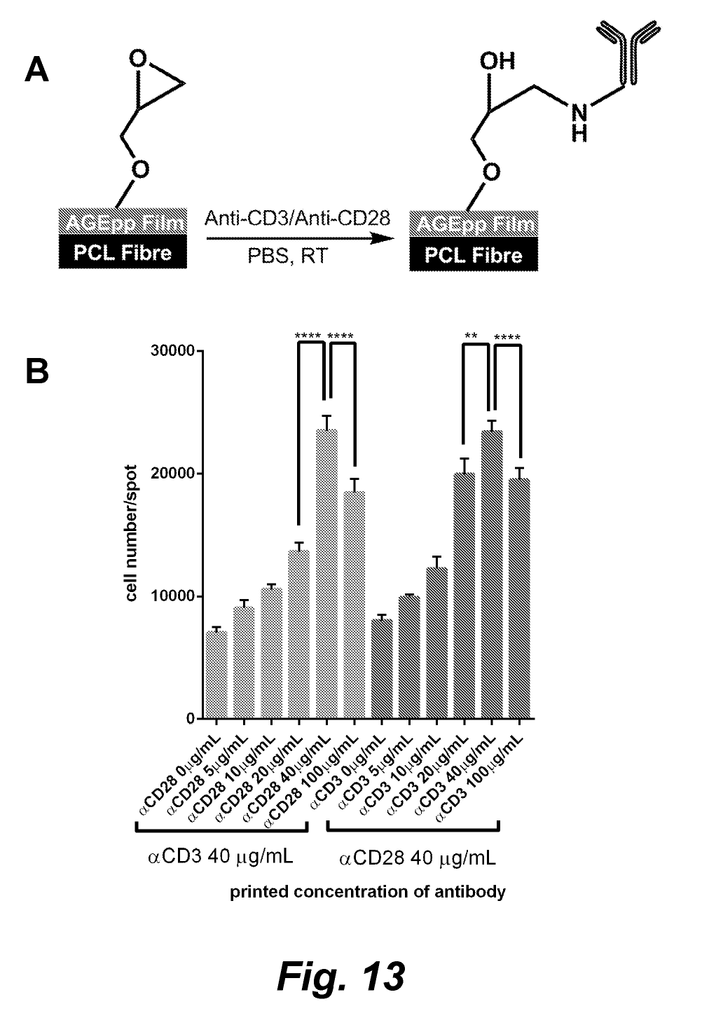

To convert the 3D scaffold to a T cell expansion platform, anti-CD3 and anti-CD28 antibodies were covalently bound to the mPCL surface. Antibody immobilisation was achieved using a two-step process: coating the fibres with a thin layer of allyl glycidyl ether plasma polymer to produce epoxy functional groups on the surface, followed by reaction of the oxirane rings in the plasma polymer with pendant amino groups of the antibody (FIG. 13A). The ratio of immobilised anti-CD3 antibody and anti-CD28 antibody found to be desirable under the conditions tested for T cell expansion was determined by cell microarray (FIG. 13B).

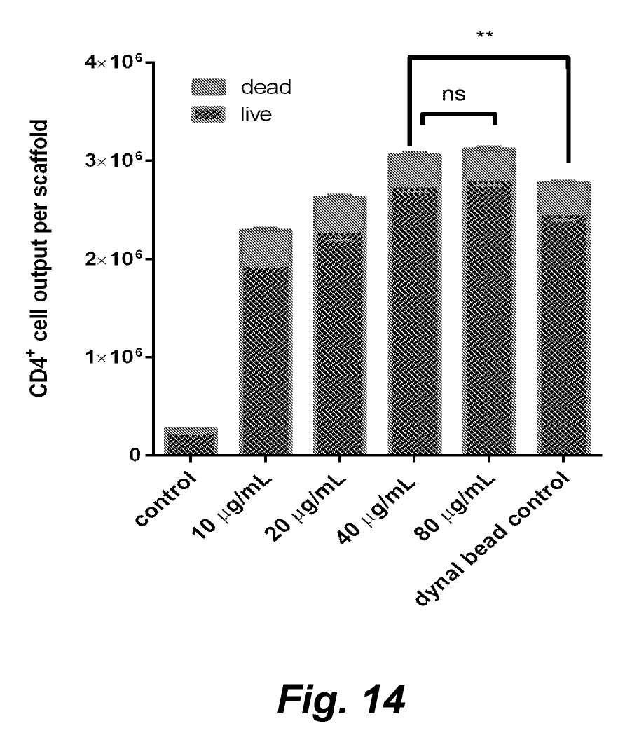

To determine the stimulation response by CD4 T-cells to a combined antibody mixture, antibody solutions were printed within each well of a 96 well plate in a 3×3 spot array. The combination of 40 μg/mL anti-CD3 antibody and 40 μg/mL anti-CD28 antibody was found to produce a desirable cell output and viability (FIG. 14)

Translating a subset of conditions analysed using the microarray platform, antibody loading of scaffolds was tested with concentrations ranging from 10-80 μg/mL and proliferation of human CD4⁺ T cells (>90% purity) cultured for 5 days with scaffolds or beads was monitored. Antibody-coated Dynal beads were used as benchmark. Robust proliferation of CD4⁺ T cells in co-culture with the functionalised scaffolds was observed over a range of antibody concentrations (FIG. 14), with the antibody concentration (40 μg/mL) selected for further experiments.

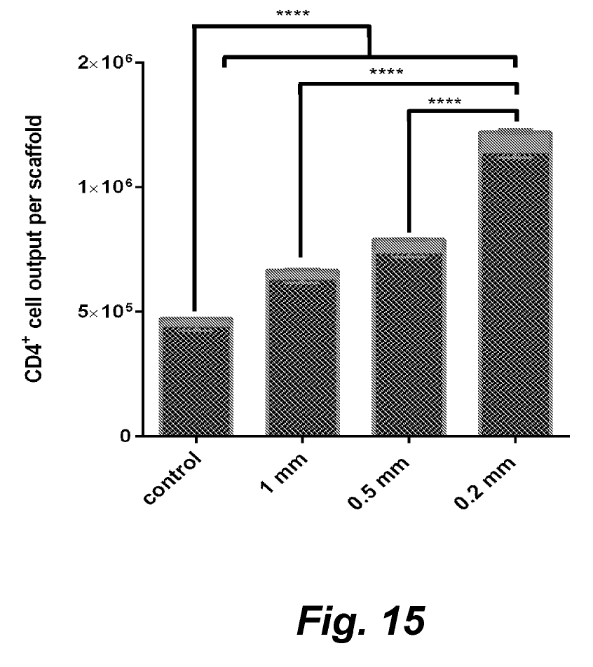

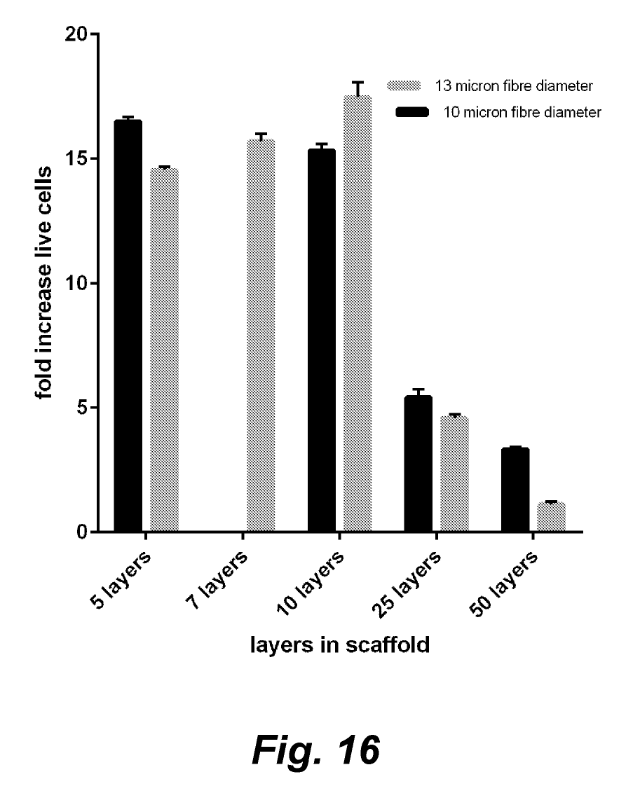

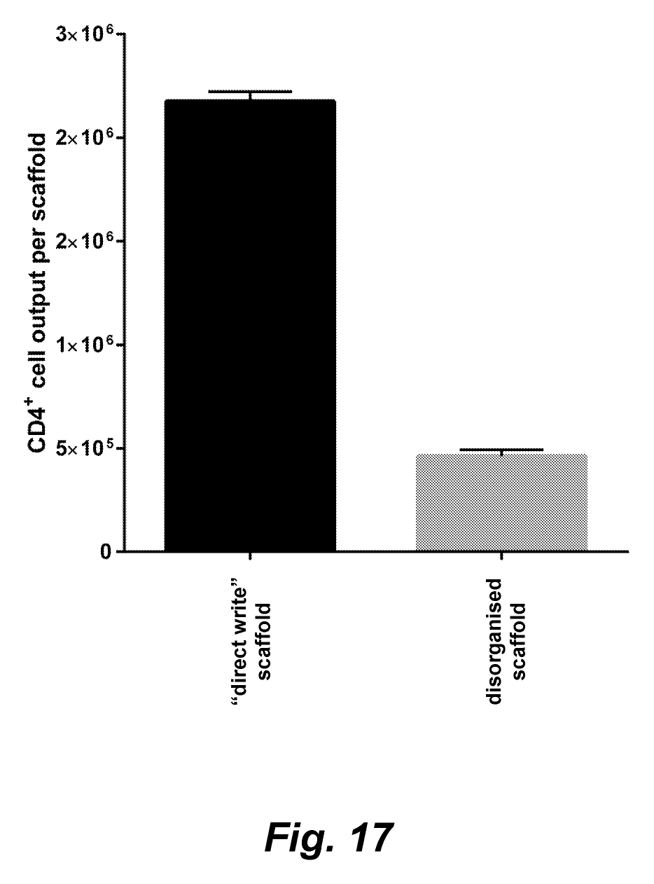

T cell expansion increased with scaffold surface area, and maximum expansion was obtained with scaffolds of 200 μm fibre spacing (FIG. 15). At this spacing, the optimal expansion was achieved with 5-20 layers in the scaffold using an average fibre diameter of 13 μm (FIG. 16). The scaffold structural formation was crucial to expansion (FIG. 17).

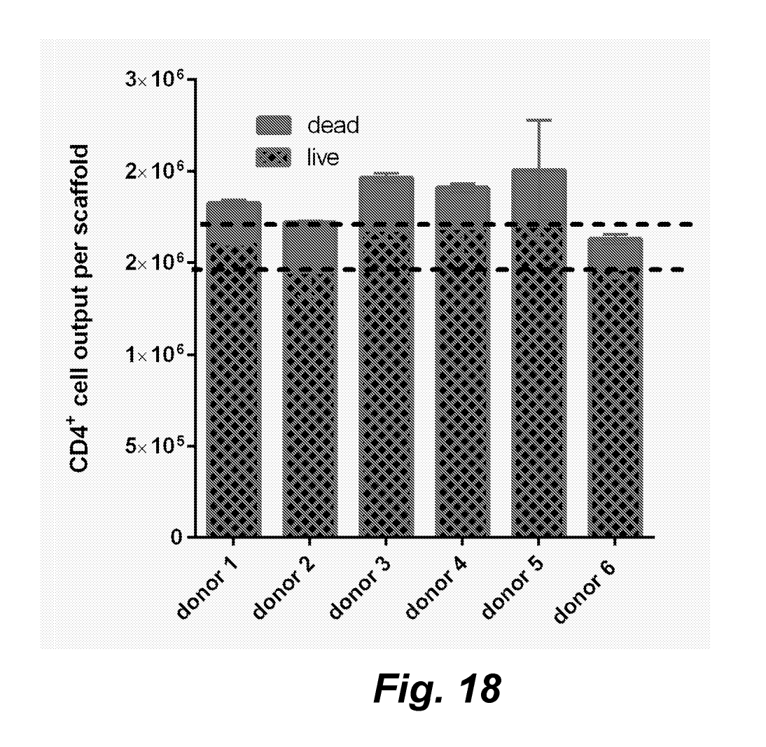

The reproducibility of the expansion yield is demonstrated by the simultaneous expansion of CD4⁺ cells isolated from six unrelated donors (FIG. 18).

Conclusions

Scaffold structural formation is critical to successful T cell expansion.

T cell expansion is desirable within the operating window of conditions tested using scaffolds of 13 micron fibre diameter, 200 micron spacing, 10-20 layers,

T cell expansion in reproducible between donors on the scaffold.

### Example 10 Expansion to Clinically Relevant Cell Numbers

Aim: To demonstrate expansion to clinical relevant cell numbers in the G-Rex expansion platform.

Methods

Scaffold Fabrication

Highly-ordered cell culture scaffolds were manufactured using a custom-made melt electrospinning writing (MEW) device. The spinning process and printer used were previously described in Example 1 with some modifications as described below. Briefly, a 5 mL glass syringe (Gastight® Cat. No. 1005, Hamilton Co., USA) was loaded with medical grade polycaprolactone (mPCL) pellets (Purasorb® PC12, Purac Biomaterials, The Netherlands). The polymer was heated up at temperatures between 90-100° C. by a coil heater with temperature controller. The syringe was connected to a programmable syringe pump (AL-1000, World Precision Instruments Inc., USA) via a wooden adapter to extrude the molten polymer through a 23G Luer lock metal needle at a constant flow rate of 10 μm/h. A high voltage between 10.5 and 12.5 kV was applied to the needle (DX250R, EMCO High Voltage Co., USA) and a grounded stainless steel collector was maintained at a distance of 20 mm to the needle. When the extruded melted polymer flew through the spinneret, an electrostatically drawn jet was accelerated toward the grounded collector. A motorised XY positioning slide (Velmex Inc., USA) driven by a CNC controller software (Mach3, Newfangled Solutions, USA) was used to displace the collector with programmable patterns to collect scaffolds of defined fibre spacing and orientation: 200 μm, 500 μm and 1000 μm fibre spacing and a lay-down pattern of 0°/90°. Scaffolds were cut to size using a laser-cutting machine (ILS12.75, Universal Laser Systems, Inc. USA).

Disorganised scaffolds were electrospun on the same apparatus and created with the same porosity as the 200 micron direct write scaffolds.

Surface Functionalisation

Scaffolds were coated with a plasma polymer to produce an epoxy-functionalised surface. The custom-built plasma rig and its operation are described in Example 1. Allyl glycidyl ether (>99%) was obtained from Sigma-Aldrich (USA). The plasma deposition protocol utilised 2 steps of plasma polymerisation, as follows: Step 1: constant wave plasma polymerisation (cw) for 1 min; Step 2: pulsed plasma polymerisation (DC, i.e. duty cycle 1 ms/20 ms) for 2 min. The pressure of the allyl glycidyl ether monomer during the treatment was at 0.2 Torr; plasma power was 25 W.

After plasma polymerisation, scaffolds were transferred to 48 well tissue culture-treated plates for antibody conjugation. Scaffolds were incubated in a solution containing a 1:1 ratio of 40 μg/μL anti-human CD3 functional grade purified antibody (Affymetrix eBioscience, USA) Cat. No: 16-0039-81, functional grade purified) and 40 μg/μL anti-human CD28 functional grade purified antibody (Becton Dickinson, USA) Cat. No: 555725, NA/LE CD28.2 functional grade purified)) in Dulbecco's phosphate buffered saline (PBS, Sigma-Aldrich) at 4° C. overnight. Scaffolds were produced using solutions containing 10, 20, 40 and 80 μg/mL of each antibody. Antibody-conjugated scaffolds were rinsed in copious PBS before use.

Human T Cell Isolation

Fresh buffy coats were obtained from the Australian Red Cross with ethics approval for research use (ARC13317). Enrichment of CD4⁺ lymphocytes was achieved by RosetteSep (Stem Cell Technologies, Australia) negative selection, as per the manufacturer's protocol. Buffy coat cells were transferred into a sterile tissue culture flask and RosetteSep human CD4⁺ T Cell enrichment cocktail added at 1 mL per 50 mL of buffy coat. The mixture was incubated with agitation using an orbital mixer for 20 min at room temperature, at approx. 300 rpm. Cells were then diluted with PBS (GE Healthcare Life Sciences, USA)+2% fetal bovine serum (FBS, Thermo Fisher Scientific, USA) at a 1:2 volume:volume ratio and mixed gently. Ficoll-Paque (15 mL, density 1.077±0.001 g/mL (+20° C.), GE Healthcare Life Sciences) was placed in a 50 mL Falcon tube, and 35 mL of the diluted blood slowly layered over the Ficoll-Paque to avoid mixing. Density sedimentation was used to isolate the CD4⁺ cell population by centrifuging the RosetteSep treated buffy coat through the Ficoll-Paque for 25 min at 400 g at room temperature, with no deceleration brake. Enriched CD4⁺ cells were removed from the plasma: ficoll interface into a new tube and washed twice with PBS+2% FBS. Isolated CD4⁺ T cells were maintained in X-Vivo 15 medium with gentamicin and phenol red (Lonza, Switzerland) completed with 5% human serum (Sigma-Aldrich), 20 nM HEPES (Thermo Fisher Scientific), 2 mM L-glutamine (Sigma-Aldrich), and IL-2 (500 U/mL, Novartis Vaccines and Diagnostics, USA) prior to use (complete X-Vivo 15 medium). CD4⁺ lymphocytes were seeded onto scaffolds within 72 h of isolation.

T Cell Expansion

T cell subsets isolated as described above were cultured in contact with antibody coated scaffolds of 3.4 cm diameter placed in G-Rex 10 (10 cm² basal surface area) single-use reactors. Reactors were inoculated at 2.5×10⁶ CD4⁺ cells in total in a medium volume of 40 mL. Culture medium is as detailed above, with the exception of IL-2 concentration (100 U/mL). A complete medium change was performed at days 8 and 12. Cultures were terminated at 15 days. For comparison, donor-matched cells were also cultured at the same starting density with 1:1 Dynabeads® Human T-Activator CD3/CD28 beads (Thermo Fisher Scientific), to establish relative stimulation compared with the bead-based antibody display.

Results

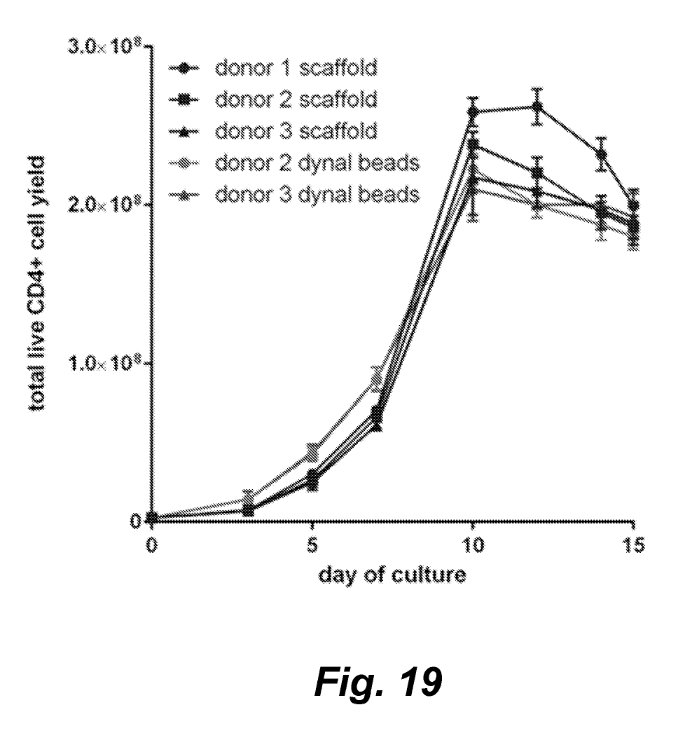

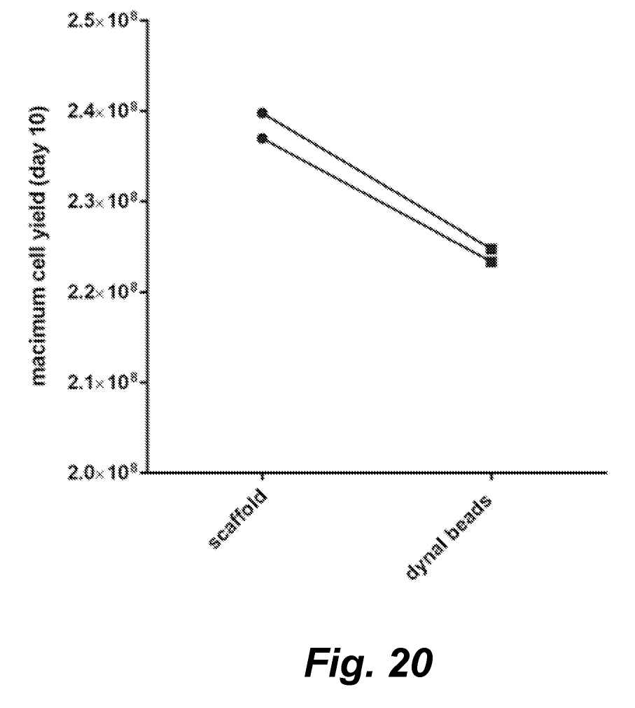

Expansion of CD4⁺ T cells at larger scale using G-Rex10 reactor yielded a 100 fold in cell number over 10 days, resulting in 2×10⁸ cell output (FIG. 19). Expansion yield was enhanced compared to bead cultures (FIG. 20).

### CONCLUSIONS

The scaffold is capable of interface with a commercial reactor system to produce a clinical relevant T cell numbers.

Although the present disclosure has been described with reference to particular examples, it will be appreciated by those skilled in the art that the disclosure may be embodied in many other forms.

Reference to any prior art in this specification is not, and should not be taken as, an acknowledgment or any form of suggestion that this prior art forms part of the common general knowledge in any country.

As used herein, the singular forms “a,” “an,” and “the” may refer to plural articles (i.e., “one or more,” “at least one,” etc.) unless specifically stated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “about” or “approximately” means an acceptable error for a particular value, which depends in part on how the value is measured or determined. In certain embodiments, “about” can mean one or more standard deviations. In certain embodiments, “about” can mean+/−20% of the stated value, or +/−10% of the stated value, or +/−5% of the stated value. It will be understood that any range stated herein that does not specifically recite the term “about” before the range or before any value within the stated range inherently includes such term to encompass the approximation within the deviation noted above.

The subject headings used herein are included only for the ease of reference of the reader and should not be used to limit the subject matter found throughout the disclosure or the claims. The subject headings should not be used in construing the scope of the claims or the claim limitations.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A porous scaffold comprising one or more conjugated T cell stimulatory molecules.

2. The porous scaffold according to claim 1, wherein the one or more T cell stimulatory molecules comprise a binding molecule for one or more of CD3, CD28, CD5, CD2, CD44, CD137, CD9, CD278, an integrin alpha and or an integrin beta.

3. The porous scaffold according to claim 1 or 2, wherein the one or more T cell stimulatory molecules comprise a binding molecule for CD3 and/or a binding molecule for CD28.

4. The porous scaffold according to any one of claims 1 to 3, wherein the one or more stimulatory molecules comprise an antibody.

5. The porous scaffold according to any one of claims 1 to 4, wherein the one or more T cell stimulatory molecules comprise an anti-CD3 antibody and/or an anti-CD28 antibody.

6. The porous scaffold according to claim 1, wherein the one or more T cell stimulatory molecules comprise fibronectin and/or a fragment or derivative thereof.

7. The porous scaffold according to any one of claims 1 to 6, wherein the porous scaffold comprises an average pore size of greater than 100 μm.

8. The porous scaffold according to any one of claims 1 to 7, wherein the porous scaffold comprises an average pore size in the range from 100 μm to 1 mm.

9. The porous scaffold according to any one of claims 1 to 7, wherein the porous scaffold comprises an average pore size of about 200 μm.

10. The porous scaffold according to any one of claims 1 to 9, wherein the porous scaffold comprises a fibrous scaffold.

11. The porous scaffold according to claim 10, wherein the fibrous scaffold comprises an average fibre spacing of greater than 100 μm.

12. The porous scaffold according to claim 10 or 11, wherein the fibrous scaffold comprises fibres with an average diameter of 5 to 20 μm.

13. The porous scaffold according to any one of claims 10 to 12, wherein the fibrous scaffold comprises 5 to 20 layers.

14. The porous scaffold according to any one of claims 10 to 13, wherein the fibrous scaffold comprises an ordered arrangement of fibres.

15. The porous scaffold according to any one of claims 1 to 14, wherein the porous scaffold comprises a mesh, a mat, a woven matrix and/or a sponge.

16. The porous scaffold according to any one of claims 1 to 15, wherein the porous scaffold comprises melt electrospun fibres.

17. The porous scaffold according to any one of claims 1 to 16, wherein the porous scaffold comprises one or more of a polylactide polymer, a polyglycolic acid polymer, a polycaprolactone polymer, a poly (amino acid alkyl ester) phosphazene polymer, a poly(caprolactone co-ethyl ethylene phosphate) polymer, a polycarbonate polymer, a polyethyleneimine polymer, a polyethyleneglycol polymer, a polyurethane polymer, and a poly vinyl alcohol polymer.

18. The porous scaffold according to any one of claims 1 to 17, wherein the one or more T cell stimulatory molecules are directly or indirectly covalently linked to the porous scaffold via a plasma polymerised functional group.

19. The porous scaffold according to claim 18, wherein the plasma polymerised functional group comprises a plasma polymerised epoxy group.

20. A method of activating a T cell, the method comprising exposing a T cell to a porous scaffold according to any one of claims 1 to 16 and thereby activating the T cell.

21. The method according to claim 20, wherein the T cell comprises a CD4⁺ T cell, a CD8⁺ T cell (a killer T cell), a CD3+ T cell, a CD4⁺CD25⁺ T cell (a regulatory T cell), a chimeric antigen receptor expressing T cell, a natural killer cell or a tumour infiltrating lymphocyte

22. A T cell activated by the method according to claim 20 or 21.

23. A method of expanding a T cell, the method comprising exposing a T cell to a porous scaffold comprising one or more conjugated T cell stimulatory molecules and culturing the T cell so as to expand the T cell.

24. The method according to claim 23, wherein the one or more T cell stimulatory molecules comprise a binding molecule for one or more of CD3, CD28, CD5, CD2, CD44, CD137 and CD9, CD278, an integrin alpha and or an integrin beta.

25. The method according to claim 23 or 24, wherein the one or more T cell stimulatory molecules comprise a binding molecule for CD3 and/or a binding molecule for CD28.

26. The method according to any one of claims 23 to 25, wherein the one or more T cell stimulatory molecules comprise an antibody.

27. The method according to any one of claims 23 to 26, wherein the one or more T cell stimulatory molecules comprise an anti-CD3 antibody and/or an anti-CD28 antibody.

28. The method according to claim 23, wherein the one or more T cell stimulatory molecules comprise fibronectin and/or a fragment or a derivative thereof.

29. The method according to any one of claims 23 to 28, wherein the porous scaffold comprises an average pore size of greater than 100 μm.

30. The method according to any one of claims 23 to 29, wherein the porous scaffold comprises an average pore size in the range from 100 μm to 1 mm.

31. The method according to any one of claims 23 to 30, wherein the porous scaffold comprises an average pore size of about 200 μm.

32. The method according to any one of claims 23 to 31, wherein the porous scaffold comprises a mesh, a mat, a woven matrix and/or a sponge.

33. The method according to any one of claims 23 to 32, wherein the porous scaffold comprises a fibrous scaffold.

34. The method according to claim 33, wherein the fibrous scaffold comprises an average fibre spacing of greater than 100 μm.

35. The method according to claim 33 or 34, wherein the fibrous scaffold comprises fibres with an average diameter of 5 to 20 μm.

36. The method according to any one of claims 33 to 35, wherein the fibrous scaffold comprises 5 to 20 layers.

37. The method according to any one of claims 33 to 36, wherein the fibrous scaffold comprises an ordered arrangement of fibres.

38. The method according to any one of claims 23 to 37, wherein the porous scaffold comprises melt electrospun fibres.

39. The method according to any one of claims 23 to 38, wherein the porous scaffold comprises one or more of a polylactide polymer, a polyglycolic acid polymer, a polycaprolactone polymer, a poly (amino acid alkyl ester) phosphazene polymer, a poly(caprolactone co-ethyl ethylene phosphate) polymer, a polycarbonate polymer, a polyethyleneimine polymer, a polyethyleneglycol polymer, a polyurethane polymer, and a poly vinyl alcohol polymer.

40. The method according to any one of claims 23 to 39, wherein the one or more T cell stimulatory molecules are directly or indirectly covalently linked to the porous scaffold via a plasma polymerised functional group.

41. The method according to claim 40, wherein the plasma polymerised functional group comprises a plasma polymerised epoxy group.

42. The method according to any one of claims 23 to 41, wherein the T cell comprises a CD4⁺ T cell, a CD8⁺ T cell (a killer T cell), a CD3⁺ T cell, a CD4⁺CD25⁺ T cell (a regulatory T cell), a chimeric antigen receptor expressing T cell, a natural killer cell or a tumour infiltrating lymphocyte.

43. The method according to any one of claims 23 to 42, wherein the culturing of the T cell comprises culturing in the presence of the porous scaffold.

44. The method according to any one of claims 23 to 43, wherein the method comprises culturing the T cell, and/or expanded cells therefrom, in the presence of accessory cells and/or an exogenous growth factor.

45. The method according to claim 44, wherein the exogenous growth factor comprises IL-2.

46. The method according to any one of claims 23 to 45, wherein the method further comprises separating expanded T cells from the porous scaffold.

47. T cells expanded by the method according to any one of claims 23 to 46.

48. A composition comprising one or more T cells activated by exposing the one or more T cells to a porous scaffold comprising one or more conjugated T cell stimulatory molecules.

49. A composition comprising one or more T cells and a porous scaffold comprising one or more conjugated T cell stimulatory molecules.

50. A complex comprising a T cell bound to a porous scaffold comprising one or more conjugated T cell stimulatory molecules.

51. A kit for activating and/or expanding T cells, the kit comprising a porous scaffold according to any one of claims 1 to 19.

52. A method of producing a porous scaffold for activating a T cell, the method comprising conjugating one or more T cell stimulatory molecules to a porous scaffold.

53. The method according to claim 47, wherein the porous scaffold comprises melt electrospun fibres.

54. The method porous scaffold according to claim 52 or 53, wherein the fibrous scaffold comprises an ordered arrangement of fibres.

55. A porous scaffold produced by the method according to any one of claims 52 to 54.

56. A cell culture vessel comprising a porous scaffold according to any one of claims 1 to 19.


SEQUENCE LISTING (6 sequences)

SEQ ID NO: 1 - DNA, 17 bp, Artificial Sequence
  FOXP3 Forward Primer
atggcccagcggatgag

SEQ ID NO: 2 - DNA, 23 bp, Artificial Sequence
  FOXP3 Reverse Primer
gaaacagcacattcccagagttc

SEQ ID NO: 3 - DNA, 22 bp, Artificial Sequence
  CTLA4 Forward Primer
catgatggggaatgagttgacc

SEQ ID NO: 4 - DNA, 23 bp, Artificial Sequence
  CTLA4 Reverse Primer
tcagtccttggatagtgaggttc

SEQ ID NO: 5 - DNA, 19 bp, Artificial Sequence
  RPL13a Forward Primer
gaggttggctggaagtacc

SEQ ID NO: 6 - DNA, 21 bp, Artificial Sequence
  RPL13a Reverse Primer
cttctcggcctgtttccgtag